(19) United States
(12) Patent Application Publication
(10) Pub. No.: US 2026/0061179 A1
(43) Pub. Date: Mar. 5, 2026

(54) ASSEMBLABLE PUMP SYSTEM AND RELATED METHODS

(71) Applicant: Puzzle Medical Devices Inc., Montreal (CA)

(72) Inventors: Gabriel GEORGES, Sherbrooke (CA); Dany FILLION, Saint-Basile-Le-Grand (CA)

(21) Appl. No.: 19/384,912

(22) Filed: Nov. 10, 2025

Related U.S. Application Data
(63) Continuation of application No. PCT/CA2024/050620, filed on May 7, 2024.
(60) Provisional application No. 63/500,776, filed on May 8, 2023.

(51) Int. Cl. A61M 60/13; A61M 60/216; A61M 60/416; A61M 60/878; A61M 60/88

(52) U.S. Cl. CPC A61M 60/13; A61M 60/216; A61M 60/416; A61M 60/878; A61M 60/88

(57) ABSTRACT

An assemblable pump system includes a first pump and a second pump that has a driveline. The second pump is configured to intraluminally assemble to the first pump via a pump-to-pump coupling between the second pump and the first pump. The driveline is further configured to intraluminally move the second pump to intraluminally assemble the second pump and the first pump together. The assemblable pump system is implantable and explantable in vivo by related methods.

### CROSS-REFERENCE PARAGRAPH TO RELATED APPLICATIONS

The present application is a continuation of International Patent Application No. PCT/CA2024/050620, filed May 7, 2024, entitled “Assemblable Pump System and Related Methods”, which claims priority to U.S. Provisional Ser. No. 63/500,776, filed May 8, 2023, entitled “Assemblable Intraluminal Pump System and Methods of Implanting and Explanting the Same”, the entirety of each of which is incorporated herein by reference.

### FIELD

This disclosure relates generally to an assemblable pump system and related methods.

### BACKGROUND

Mechanical circulatory support (MCS) devices are technologies that notably support blood flow circulation in patients with heart failure. MCS therapies may be used to assist the left ventricle in the case of left ventricular assist devices (LVAD), the right ventricle in the case of right ventricular assist devices (RVAD), or both ventricles in the case of biventricular assist devices (BiVAD). These devices may be divided into two categories: temporary circulatory support devices for indications such as high-risk percutaneous interventions (PCIs), and long-term support devices for indications such as bridge-to-heart transplantation, acute decompensated heart failure (ADHF), and end-stage heart failure. MCS devices may be implanted surgically and transcatheterly and, generally, are considered for patients with advanced heart failure or rapidly progressing heart failure that do not respond to medical therapy. The choice of a particular MCS therapy depends on the clinical presentation.

In order to take full advantage of non-invasive transcatheter procedures, it is generally desirable for transcatheterly implantable MCS devices to have a relatively small cross-sectional size that allow them to be routed in blood vessels of compatible lumen size. Such blood vessel may not necessarily be amenable to MCS devices of larger cross-sectional size due to size restriction. The use of MCS devices of small cross-sectional size clinically translates into lower rates of vascular access site complications, such as major bleeding, infection, and need for urgent surgery, as compared to MCS devices of larger cross-sectional sizes.

To provide sufficient blood outflow for hemodynamic support, some known MCS devices are required to be operated at relatively high speeds, for example as high as 55,000 rpm. Operation at such speeds causes supraphysiological scalar shear stresses to the blood, leading for example to blood elements destruction, hemolysis, platelet consumption and activation, as well as loss of von Willebrand Factor activity; all of which are associated with adverse outcomes. In short, at least some known MCS devices are not necessarily suited for long-term medical indications requiring implantation and operation of the devices for a relatively long period of time, during which exposure to blood damage-related complications, such as stroke and gastro-intestinal bleeding, accumulates.

Finally, known MCS devices usually require to be powered by one or more electrical wire(s) or cable(s) in order to provide hemodynamic support. The electrical wire(s) or cable(s) may represent a substantial fraction of the vessel's lumen in which the wire(s) is/are routed therealong. This is particularly the case when multiple electrical wires or cables are routed along a lumen and/or when the lumen progressively reduces in size downstream and upstream of an implanted MCS device. As a consequence, the electrical wire(s) or cable(s) may hinder blood flowing through the vasculature, resulting in deleterious physiological effects including tissue ischemia.

Therefore, improvement over at least some of the known MCS devices is desirable.

### SUMMARY

A first aspect of this disclosure is directed to an implantable pump system, including: a first pump; and a second pump that includes a driveline, the second pump is configured to intraluminally assemble to the first pump via a pump-to-pump coupling between the second pump and the first pump, the driveline is further configured to move the second pump to intraluminally assemble the second pump and the first pump together.

The first pump may include a first pumping unit, and the second pump may include a second pumping unit, the pump-to-pump coupling may include a unit-to-unit coupling between the first pumping unit and the second pumping unit.

The first pump may include a pumping unit, the pump-to-pump coupling may include a unit-to-driveline coupling between the pumping unit of the first pump and the driveline of the second pump.

The first pump may include a driveline.

The pump-to-pump coupling may include a driveline-to-driveline coupling between the driveline of the first pump and the driveline of the second pump.

The driveline of the first pump may be configured to removably attach to the first pump.

The driveline of the first pump may be configured to screw to the first pump.

The driveline of the first pump may be configured to snare the first pump.

The driveline of the first pump may include an electrical conductor that is configured to transmit electricity from a power source to the first pump for operating the same.

The driveline of the first pump may be configured to slidably move the first pump to intraluminally assemble the first pump and the second pump together.

The driveline of the first pump may be configured to be extraluminally manipulated to move the first pump to intraluminally assemble the first pump and the second pump together.

The implantable pump system may include a sleeve coupler configured to attach to the driveline of the first pump and to slidably receive the driveline of the second pump therein.

The implantable pump system may include a sleeve coupler configured to slidably receive the driveline of the first pump and the second driveline of the second pump therein.

The implantable pump system may include a coupler associated with one of the first pump and the second pump, the coupler may be configured to slidably receive the other one of the first pump and the second pump therein for intraluminally assembling the first pump and the second pump together.

The coupler may include a pump-receiving coupler defining an opening sized and shaped to receive the other one of the first pump and the second pump therein.

The coupler may be a latch coupler.

The implantable pump system may include a first physical connector associated with the first pump, and a second physical connector associated with the second pump, the first physical connector and the second physical connector may be configured to couple together for intraluminally assembling the first pump and the second pump together.

The first physical connector may include one of a male connector and a female connector, and the second physical connector may include the other one of the male connector and the female connector.

The first physical connector may be a first latch connector, and the second physical connector may be a second latch connector.

The implantable pump system may include a first magnetic connector associated with the first pump, and a second magnetic connector associated with the second pump, the first magnetic connector and the second magnetic connector may be configured to magnetically couple together for intraluminally assembling the first pump and the second pump together.

The first magnetic connector may include a first drive unit magnet, and the second magnetic connector may include a second drive unit magnet.

The implantable pump system may include a first electrical connector associated with the first pump, and a second electrical connector associated with the second pump, the first electrical connector and the second electrical connector may be configured to electrically connect together for electrically connecting the first pump and the second pump together.

At least one of the first electrical connector and the second electrical connector may include a fluid seal configured to provide a fluid tight contact upon electrical connection therebetween.

The coupler has an undeployed state for transcatheter delivery of at least one of the first pump and the second pump intraluminally, and a deployed state for intraluminally assembling the second pump and the first pump together.

The driveline of the second pump may be configured to removably attach to the second pump.

The driveline of the second pump may be configured to screw to the second pump.

The driveline of the second pump may be configured to snare the second pump.

The driveline of the second pump may include an electrical conductor configured to transmit electricity from a power source to the second pump for operating the same.

The driveline of the second pump may be configured to slidably move the second pump to intraluminally assemble the second pump and the first pump together.

The driveline of the second pump may be configured to be extraluminally manipulated to move the second pump to intraluminally assemble the second pump and the first pump together.

The implantable pump system may include an anchor attached to at least one of the first pump and the second pump, the anchor may be configured to engage a lumen wall for intraluminally anchoring the first pump and the second pump.

The anchor may be releasably attached to at least one of the first pump and the second pump.

The first and the second pumps may be intravascular pumps or any other medical devices that may be assembled to each other and implanted.

The drivelines of the first and second pumps may be control wires or control cables.

A second aspect of this disclosure is directed to a method of implanting an assemblable medical device system, such as the assemblable pump system, at an implantation site in a lumen of a subject, the method including: delivering the assemblable pump system to the implantation site, the assemblable pump system including a first pump and a second pump having a driveline, the first pump and the second pump are configured to intraluminally assemble together via a pump-to-pump coupling between the first pump and the second pump; and manipulating the driveline to move the second pump and to intraluminally assemble the second pump and the first pump together.

Delivering may include obtaining a single access opening for delivering the first pump and the second pump therethrough to the implantation site.

Delivering may include introducing a sheath, containing at least partially the first pump and the second pump therein, in the lumen through the single access opening; navigating the sheath in the lumen up to the implantation site thereof; exiting the first pump and the second pump from the sheath at the implantation site; and removing the sheath from the lumen and through the single access opening.

Delivering may include closing the single access opening on the driveline of the second pump.

Manipulating the driveline of the second pump may include at least one of pulling and pushing the driveline of the second pump through the single access opening so that the second pumping unit and the first pumping unit get closer from each other to intraluminally assemble the second pump and the first pump together.

The method of implanting may include at least one of pulling and pushing a driveline of the first pump through the single access opening so that the first pumping unit and the second pumping unit get closer from each other to intraluminally assemble the first pump and the second pump together.

At least one of manipulating the driveline of the second pump and manipulating the driveline of the first pump may include manipulating an extracorporeal end portion of at least one of the driveline of the second pump and the driveline of the first pump.

The method of implanting may include transmitting electricity to the second pump via the driveline thereof disposed through the single access opening for operating the second pump.

Manipulating the driveline of the second pump may include electrically connecting the second pump and the first pumps together to transmit electricity between the second pump and the first pump for operating the first pump, when the second pump and the first pump are assembled together.

The method of implanting may include transmitting electricity to the first pump via the driveline thereof disposed through the single access opening for operating the first pump.

Delivering may include obtaining a first access opening for delivering the first pump therethrough to the implantation site, and obtaining a second access opening for delivering the second pump therethrough to the implantation site.

Delivering may include introducing a first sheath, containing at least partially the first pump therein, in the lumen through the first access opening, and introducing a second sheath, containing at least partially the second pump therein, in the lumen through the second access opening; navigating the first sheath and the second sheath in the lumen up to the implantation site thereof; exiting the first pump from the first sheath at the implantation site thereof, and exiting the second pump from the second sheath at the implantation site thereof; and removing the first sheath from the lumen through the first access opening, and removing the second sheath from the lumen through the second access opening.

Delivering may include closing the first access opening and the second access opening.

Manipulating the driveline of the second pump may include at least one of pulling and pushing the driveline of the second pump through the second access opening so that the second pumping unit and the first pumping unit get closer from each other to intraluminally assemble the second pump and the first pump together.

The method of implanting may include at least one of pulling and pushing a driveline of the first pump through the first access opening so that the first pumping unit and the second pumping unit get closer from each other to intraluminally assemble the first pump and the second pump together.

At least one of manipulating the driveline of the second pump and manipulating the driveline of the first pump may include manipulating an extracorporeal end portion of at least one of the driveline of the second pump and the driveline of the first pump.

The method of implanting may include transmitting electricity to the second pump via the driveline thereof disposed through the second access opening for operating the second pump.

Manipulating the driveline of the second pump may include electrically connecting the second pump and the first pumps together to transmit electricity between the second pump and the first pump for operation, when the second pump and the first pump are assembled together.

The method of implanting may include transmitting electricity to the first pump via a driveline thereof disposed through the first access opening for operating the first pump.

The method of implanting may include anchoring the assemblable pump system at the implantation site.

A third aspect of this disclosure is directed to a method of explanting an assemblable medical device system, such as the assemblable pump system, which includes a first pump and a second pump, from an implantation site in a lumen of a subject, the method including: manipulating a driveline of the second pump to move the second pump and to intraluminally unassemble the second pump from the first pump that is coupled to the second pump via a pump-to-pump coupling; and retrieving the assemblable pump system that is unassembled from the implantation site.

Retrieving may include obtaining a single access opening for retrieving the first pump and the second pump therethrough from the implantation site.

Retrieving may include introducing a sheath in the lumen through the single access opening; navigating the sheath in the lumen up to the implantation site thereof; sheathing at least partially the first pump and the second pump; and removing the sheath, containing at least partially the first pump and the second pump therein, from the lumen and through the single access opening.

Retrieving may include closing the single access opening.

Manipulating the driveline of the second pump may include at least one of pulling and pushing the driveline of the second pump through the single access opening so that the second pumping unit and the first pumping unit move away from each other to intraluminally unassemble the second pump and the first pump from each other.

The method of explanting may include at least one of pulling and pushing a driveline of the first pump through the single access opening so that the first pumping unit and the second pumping unit move away from each other to intraluminally unassemble the first pump and the second pump from each other.

At least one of manipulating the driveline of the second pump and manipulating the driveline of the first pump may include manipulating an extracorporeal end portion of at least one of the driveline of the second pump and the driveline of the first pump.

The method of explanting may include stopping transmitting electricity to the second pump via the driveline thereof disposed through the single access opening.

Manipulating the driveline of the second pump may include electrically disconnecting the second pump and the first pumps from each other to stop transmitting electricity between the second pump and the first pump.

The method of explanting may include stopping transmitting electricity to the first pump via the driveline thereof disposed through the single access opening.

Retrieving may include obtaining a first access opening for retrieving the first pump therethrough from the implantation site, and obtaining a second access opening for retrieving the second pump therethrough from the implantation site.

Retrieving may include introducing a first sheath in the lumen through the first access opening, and introducing a second sheath in the lumen through the second access opening; navigating the first sheath and the second sheath in the lumen up to the implantation site thereof; sheathing the first pump in the first sheath, and sheathing the second pump in the second sheath; and exiting the first sheath, containing at least partially the first pump therein, from the lumen and through the first access opening, and exiting the second sheath, containing at least partially the second pump therein, from the lumen and through the second access opening.

Retrieving may include closing the first access opening and the second access opening.

Manipulating the driveline of the second pump may include at least one of pulling and pushing the driveline of the second pump through the second access opening so that the second pumping unit and the first pumping unit move away from each other to intraluminally unassemble the second pump and the first pump from each other.

The method of explanting may include at least one of pulling and pushing a driveline of the first pump through the first access opening so that the first pumping unit and the second pumping unit move away from each other to intraluminally unassemble the first pump and the second pump from each other.

At least one of manipulating the driveline of the second pump and manipulating the driveline of the first pump may include manipulating an extracorporeal end portion of at least one of the driveline of the second pump and the driveline of the first pump.

The method of explanting may include stopping transmitting electricity to the second pump via the driveline thereof disposed through the second access opening for stopping operating the second pump.

Manipulating the driveline of the second pump may include electrically disconnecting the second pump and the first pumps from each other to stop transmitting electricity between the second pump and the first pump, when the second pump and the first pump are unassembled from each other.

The method of explanting may include stopping transmitting electricity to the first pump via a driveline thereof disposed through the first access opening for stopping operating the first pump.

The method of explanting may include unanchoring the assemblable pump system from the implantation site.

### Definitions

Throughout the description and claims, the word “comprising”, “including”, “having”, and variations of these words are to be construed as open-ended terms and, thus, are not intended to exclude other elements, limitations, technical, additives, components, steps, and the like.

The expression “and/or” is intended herein to mean “one or more” of the elements so combined, i.e., elements that are conjunctively present in some cases and disjunctively present in other cases. Whether related or unrelated to the elements specifically identified, other elements may optionally be present other than the elements specifically identified by the expression “and/or. Therefore, as a non-limiting example, a reference to an “element A” and/or an “element B” can refer to the “element A” only (optionally including element(s) other than the “element B”), to the “element B” only (optionally including element(s) other than the “element A”), or to both the “element A” and the “element B” (optionally including element(s) other than the “element A”and the “element B”).

Relational terms such as first and second, top and bottom, and the like may be used solely to distinguish one entity or action from another entity or action without necessarily requiring or implying any actual such relationship or order between such entities or actions. The description may use perspective-based descriptions such as up/down, back/front, and top/bottom. Such descriptions are merely used to facilitate the discussion and are not intended to restrict the application of disclosed embodiments. Various operations may be described as multiple discrete operations in tum, in a manner that may be helpful in understanding embodiments; however, the order of description should not be construed to imply that these operations are order dependent.

The terms “about” and/or “approximately” when used in conjunction with values and/or ranges generally refer to those values and/or ranges near to a recited value and/or range. The terms “about” and “approximately” may be used interchangeably. These terms generally refer to a range of numbers that one of skill in the art would consider equivalent to the recited values (i.e., having the same function or result). In many instances these terms may include numbers that are rounded to the nearest significant figure.

The term “substantially” when used in conjunction with physical and/or geometric feature(s), structure(s), characteristic(s), relationship(s), etc. is intended to convey that the feature(s), structure(s), characteristic(s), relationship(s), etc. so defined is/are nominally the feature(s), structure(s), characteristic(s), relationship(s), etc. Generally, the use of “substantially” herein is intended to account that, although equality may be desirable, some variance may occur. Such variance may result from manufacturing tolerances, limitations, approximations, and/or other practical considerations.

The terms “wire(s)” and “cable(s)” are used herein without distinction to refer to an elongated structure capable of being introduced and used (e.g., to slidably move) intraluminally, such as intravascularly. Example of “elongated structure” includes guide wires as well as any other cables and wires, either it be configured to transmit electricity or not.

The terms “proximal” and “proximally” refer to a location or a position that is closer to an operator, as compared to a location or a position that is distal to, or distally positioned or distally located to, this operator.

The terms “distal” and “distally” refer to a location or a position that is farther to an operator, as compared to a location or a position that is proximal to, or proximally positioned or proximally located to, this same operator.

Herein various embodiments of the systems and methods are described. In many of the different embodiments, features are similar. Therefore, to avoid redundancy, repetitive descriptions of these similar features may not be made in some circumstances. It shall be understood, however, that description of a first-appearing feature applies to the later described similar feature and each respective description, therefore, is to be incorporated therein without such repetition.

### BRIEF DESCRIPTION OF THE DRAWINGS

In order that this disclosure be readily understood, at least some selected embodiments thereof are illustrated by way of example(s) in the accompanying drawings. Accordingly, the accompanying drawings are illustrative in nature only and are not intended to be construed and interpreted as limiting the extent of the subject-matter protected by the claims.

It is noted that like reference numerals identify similar or equivalent elements and/or features throughout the drawings. If present in the claims, reference numerals are provided only to make claims easier to comprehend and are not intended to be construed and interpreted as limiting the extent of the subject-matter protected by the claims. The elements and/or features illustrated throughout the drawings are not necessarily drawn to scale.

To help understand the drawings, item(s), component(s), and box(es) that is/are optional may be generally represented by dashed or dotted lines.

FIGS. 1-2 are schematic representations of assemblable pump systems that include first and second pumps with only one of them provided with a corresponding coupler, in accordance with some embodiments of a first aspect of this disclosure. The first and second pumps are represented in an assembled state.

FIG. 3 is a schematic representation of an assemblable pump system that includes a first pump having a first coupler, and a second pump having a second coupler, in accordance with an embodiment of the first aspect of this disclosure. The first and second pumps are represented in an assembled state.

FIGS. 4-6 are schematic representations of various coupling modes between the first and second pumps of the assemblable pump system of FIGS. 1-3, in accordance with some embodiments.

FIGS. 7-9 are schematic representations of a unit-to-unit coupling mode between the first and second pumps of the assemblable pump system of FIGS. 1-3, in accordance with some embodiments.

FIGS. 10-12 are schematic representations of a unit-to-driveline coupling mode between the first and second pumps of the assemblable pump system of FIGS. 1-3, in accordance with some embodiments.

FIGS. 13-15 are schematic representations of a driveline-to-driveline coupling mode between the first and second pumps of the assemblable pump system of FIGS. 1-3, in accordance with some embodiments.

FIGS. 16-17 are schematic representations of the assemblable pump system of FIGS. 1-3 with the first and second pumps in an unassembled state and an assembled state, respectively, in accordance with some embodiments.

FIGS. 18-21 are schematic representations of the assemblable pump system of FIGS. 1-3 with the first and second pumps in an assembled state and having various relative positions, in accordance with some embodiments.

FIGS. 22-24 are schematic representations of the cross-sectional size of the assemblable pump system of FIGS. 1-3 with the first and second pumps in unassembled and assembled states, in accordance with some embodiments. FIG. 23 is taken along the plane XXIII-XXIII of FIG. 22.

FIGS. 25-26 are schematic representations of the assemblable pump system of FIGS. 1-3 showing the conversion of the first and second pumps between unassembled and assembled states, in accordance with some embodiments.

FIGS. 27-28 are schematic representations of the assemblable pump system of FIGS. 1-3 provided with one or two anchors, respectively, in accordance with some embodiments.

FIGS. 29-30 are schematic representations of the assemblable pump system of FIGS. 1-3 provided with three or four pumps, respectively, in accordance with some embodiments.

FIGS. 31-33 are perspective and side views of an assemblable pump system, such as one of FIGS. 1-3, that includes a pump-receiving coupler on a first pumping unit of a first pump thereof, in accordance with one or more embodiments.

FIGS. 34-36 are perspective and side views of an assemblable pump system, such as one of FIGS. 1-3, that includes an anchor having a pump-receiving coupler, in accordance with one or more embodiments.

FIGS. 37-38 are side views of an assemblable pump system, such as one of FIGS. 1-3, that includes a pump-receiving coupler on a driveline of a first pump thereof and an optional magnetic coupler on each of first and second pumping units, in accordance with one or more embodiments.

FIGS. 39-41 are perspective and side views of an assemblable pump system, such as one of FIGS. 1-3, that includes a male connector on a first pumping unit, and a female connector on a second pumping unit, in accordance with one or more embodiments.

FIGS. 42-43 are perspective and side views of an assemblable pump system, such as one of FIGS. 1-3, that includes a first magnetic connector on a first pump, and a second magnetic connector on a second pump, in accordance with one or more embodiments.

FIGS. 44-45 are perspective and side views of an assemblable pump system, such as one of FIGS. 1-3, that includes a first electrical connector on a first pumping unit, and a second electrical connector on a second pumping unit, in accordance with one or more embodiments.

FIGS. 46-48 are side views of an assemblable pump system that includes a sleeve coupler, in accordance with one or more embodiments.

FIGS. 49-50 are side views of an assemblable pump system that includes a butterfly snare coupler, in accordance with one or more embodiments.

FIG. 51 is a schematic representation of a method of implanting an assemblable pump system in vivo, in accordance with a second aspect of this disclosure.

FIG. 52 is a schematic representation of an assemblable pump system that is implanted in vivo, such as by the method of FIG. 51, via a single access opening, in accordance with one or more embodiments.

FIG. 53 is a schematic representation of an assemblable pump system that is implanted in vivo, such as by the method of FIG. 51, via first and second access openings, in accordance with one or more embodiments.

FIG. 54 is a schematic representation of a method of explanting an assemblable pump system in vivo, in accordance with a third aspect of this disclosure.

### DETAILED DESCRIPTION

The subject-matter of this disclosure is described and explained in the following detailed description with reference to the non-limiting aspect(s), embodiment(s), example(s), feature(s), element(s), step(s), and/or other piece(s) of information presented herein and illustrated in the accompanying non-limiting drawings and/or figures, as the case may be. Recognizing that the foregoing may vary, the skilled addressee shall readily appreciate that any other variants thereof as well as any combination of these other variants are contemplated without departing from the scope of this disclosure, even if not explicitly disclosed herein.

The explicit and implicit content of this disclosure are intended merely for the purpose of understanding one or more ways in which the subject-matter claimed herein may be reduced to practice by the skilled addressee. Therefore, the explicit and implicit content of this disclosure shall not to be construed as limiting the scope of the subject-matter claimed herein which scope is defined solely by the accompanying claims and applicable law.

Similarly, the terminology used herein is merely for the purpose of describing and explaining the subject-matter claimed and is not intended to limit the scope thereof. Unless defined otherwise, all technical, engineering, scientific, and other relevant terminology used herein have the same meanings as commonly understood by the skilled addressee.

With the foregoing in mind, this disclosure relates generally to an MCS device, namely an assemblable pump system, that includes a plurality of pumps (also referred to herein as “implantable pumps” or “intraluminal pumps” or “transcatheter pumps”), such as a first pump and a second pump. The assemblable pump system is convertible from an unassembled state to an assembled state and/or from assembled state to an unassembled state. In the unassembled state, the assemblable pump system can be navigated in, delivered and implanted in, as well as retrieved and explanted from a subject's body. In the assembled state the first and second pumps are assembled to each other, and the assemblable pump system may be operated, for example, to produce a hemodynamic effect in the subject's body. The assemblable pump system can also be operated in the unassembled state. The assemblable pump system can be used in a subject's body by transcatheter approaches.

FIGS. 1-3 schematically illustrate various states of an assemblable pump system 100, according to embodiments.

FIG. 1 schematically illustrates the assemblable pump system 100 that includes a first pump 102, which is provided with a first pumping unit 104 and a coupler 114, as well as a second pump 122, which is provided with a second pumping unit 124 and a driveline 130 (may also be referred to herein as a “control wire”) that is configured to transmit power to the second pumping unit 124 for operating the same, with the first and second pumps 102, 122 assembled to each other in an assembled state via the coupler 114, according to one or more embodiments. The coupler 114 may be provided to the first pumping unit 104 and/or the driveline 110 (may also be referred to herein as a “control wire”) of the first pump 102. The coupler 114 may be configured to reversibly or non-reversibly couple the second pumping unit 124 and/or the driveline 130 of the second pump 122 to assemble the first and second pumps 102, 122 to each other in the assembled state. The coupler 114 may be configured to transfer power between the first and second pumps 102, 122 for operating the first pumping unit 104 when the first and second pumps 102, 122 are in the assembled state. The first pump 102 may optionally include a driveline 110 that is configured to provide power to the first pumping unit 104 for operating the same. So, power may be supplied to the first pumping unit 104 through the coupler 114 and/or the driveline 110.

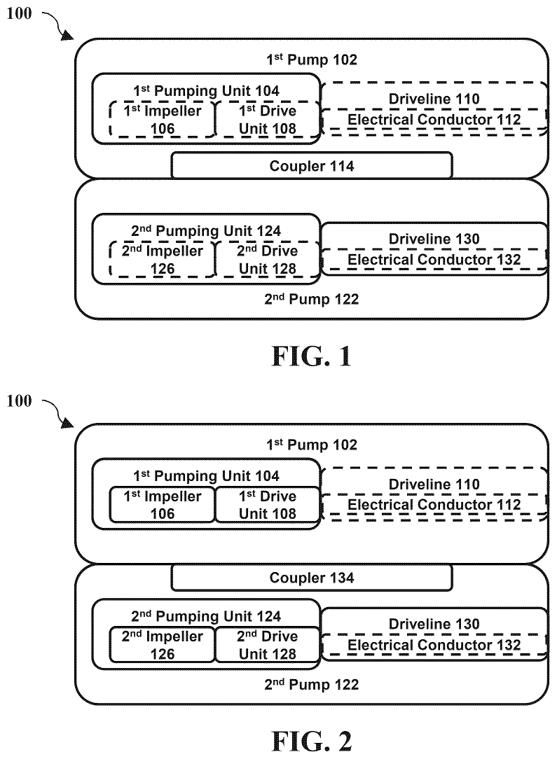

FIG. 2 schematically illustrates the assemblable pump system 100 that includes the first pump 102, which is provided with the first pumping unit 104, as well as the second pump 122, which is provided with the second pumping unit 124 and the driveline 130 that is configured to transmit power to the second pumping unit 124 for operating the same, with the first and second pumps 102, 122 assembled to each other in an assembled state via the coupler 134, according to one or more embodiments. The coupler 134 may be provided to the second pumping unit 124 and/or the driveline 130 of the second pump 122. The coupler 134 may be configured to reversibly or non-reversibly couple the first pumping unit 104 and/or the driveline 110 of the first pump 102 to assemble the first and second pumps 102, 122 to each other in the assembled state. The coupler 134 may be configured to transfer power between the first and second pumps 102, 122 for operating the first pumping unit 104 when the first and second pumps 102, 122 are in the assembled state. The first pump 102 may optionally include a driveline 110 that is configured to transmit power to the first pumping unit 104 for operating the same. So, power may be supplied to the first pumping unit 104 through the coupler 134 and/or the driveline 110.

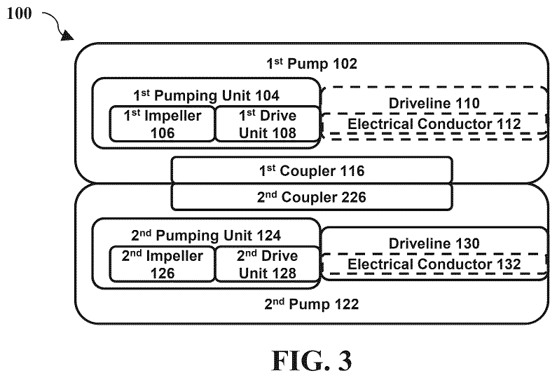

FIG. 3 schematically illustrates the assemblable pump system 100 that includes the first pump 102, which is provided with the first pumping unit 104 and a first coupler 116, as well as the second pump 122, which is provided with the second pumping unit 124, a second coupler 226, and the driveline 130 that is configured to transmit power to the second pumping unit 124 for operating the same, with the first and second pumps 102, 122 assembled to each other in an assembled state via the first and second couplers 116, 226, according to one or more embodiments. The first coupler 116 may be provided to the first pumping unit 104 and/or the driveline 110 of the first pump 122. The first coupler 116 may be configured to reversibly or non-reversibly couple the second pumping unit 124, the driveline 130, and/or the second coupler 226 of the second pump 122 to assemble the first and second pumps 102, 122 to each other in the assembled state. The second coupler 226 may be provided to the second pumping unit 124 and/or the driveline 130 of the second pump 122. The second coupler 226 may be configured to reversibly or non-reversibly couple the first pumping unit 104, the driveline 110, and/or the first coupler 116 of the first pump 102 to assemble the first and second pumps 102, 122 to each other in the assembled state. One of both of the first and second couplers 116, 226 may be configured to transfer power between the first and second pumps 102, 122 for operating the first pumping unit 104 when the first and second pumps 102, 122 are in the assembled state. The first coupler 116 may be the coupler 114, and the second coupler 226 may be the coupler 134. The first pump 102 may optionally include a driveline 110 that is configured to transmit power to the first pumping unit 104 for operating the same. So, power may be supplied to the first pumping unit 104 through the first coupler 116, the second coupler 226, and/or the driveline 110.

Referring to FIGS. 1-3, conversion of the assemblable pump system 100 between the assembled state and an unassembled state, in which the first and second pumps 102, 122 are not assembled to each other via the couplers 114, 134, 116, 226, may be achieved by manipulation of the driveline 130 and/or the driveline 110 (when present), according to embodiments. The assemblable pump system 100 may be converted from the assembled state to the unassembled state and/or from unassembled state to the assembled state. In particular, pulling one of the drivelines 110, 130, while optionally pushing the other one of the drivelines 110, 130, may move at least one of the first and second pumps 102, 122 toward the other one of the first and second pumps 102, 122 and convert the assemblable pump system 100 from the unassembled state to the assembled state. Pushing one of the drivelines 110, 130, while optionally pulling the other one of the drivelines 110, 130, may move at least one of the first and second pumps 102, 122 toward the other one of the first and second pumps 102, 122 and convert the assemblable pump system 100 from the unassembled state to the assembled state. Pulling one of the drivelines 110, 130, while optionally pushing the other one of the drivelines 110, 130, may move at least one of the first and second pumps 102, 122 away from the other one of the first and second pumps 102, 122 and convert the assemblable pump system 100 from the assembled state to the unassembled state. Pushing one of the drivelines 110, 130, while optionally pulling the other one of the drivelines 110, 130, may move at least one of the first and second pumps 102, 122 away from the other one of the first and second pumps 102, 122 and convert the assemblable pump system 100 from the assembled state to the unassembled state.

A portion of one or both of the drivelines 110, 130 that is disposed outside a subject's body (i.e., “extracorporeally” disposed) may be manipulated to convert between the assembled and unassembled states the assemblable pump system 100 that is located inside a subject's body (i.e., “intracorporeally” disposed). For example, the assemblable pump system 100 may be located in a lumen of the subject body (i.e., “intraluminally” disposed). As such, the assemblable pump system 100 may be converted between the assembled and unassembled states at an implantation site inside the subject's body.

Still referring to FIGS. 1-3, the first and second pumps 102, 122 may be impeller pumps and therefore the first and second pumping units 104, 124 may include first and second impellers 106, 126, respectively, for imparting movement to a fluid, such as blood, upon rotation, according to one or more embodiments. As illustrated, the first and second pumps 102, 122 may also include first and second drive units 108,128, such as electric motors, configured to rotate the first and second impellers 106, 126, respectively. Also, to power the first and second drive units 108,128, the first and second drivelines 110, 130 may include electrical conductors 112, 132 configured to transmit electricity from a power source to the first and second drive units 108,128, respectively.

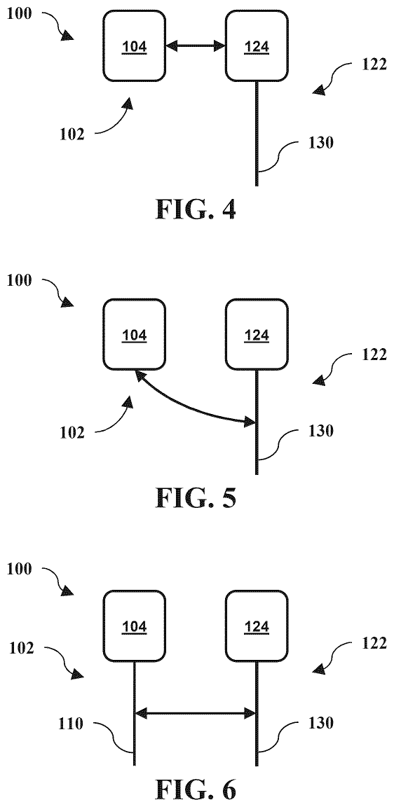

FIGS. 4-6 illustrate various modes of pump-to-pump couplings (as illustrated by the double-headed arrow) between the first and second pumps 102, 122, according to some embodiments. In particular, FIG. 4 illustrates the first and second pumps 102, 122 coupling to each other via the first and second pumping units 104, 124, respectively (also referred to herein as a unit-to-unit coupling), according to one or more embodiments. FIG. 5 illustrates the first and second pumps 102, 122 coupling to each other by the first pumping unit 104 and the driveline 130, respectively (also referred to herein as a unit-to-driveline coupling), according to one or more embodiments. FIG. 6 illustrates the first and second pumps 102, 122 coupling to each other by the drivelines 110, 130, respectively (also referred to herein as a driveline-to-driveline coupling), according to one or more embodiments.

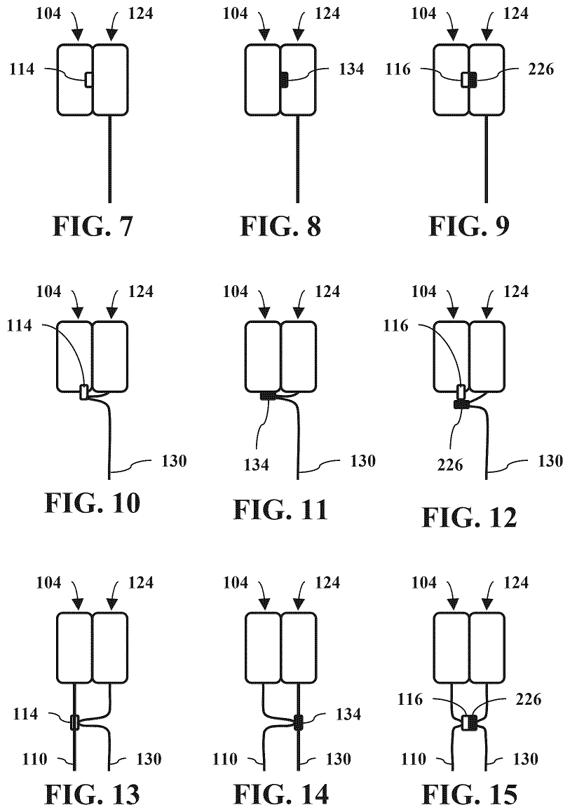

FIGS. 7-15 illustrate various arrangements of the couplers 114, 134 as well as various arrangements of the first and second couplers 116, 226 associated with the first and second pumps 102, 122, according to embodiments.

With reference to FIG. 4, the first and second pumps 102, 122 couple to each other by the first and second pumping units 104, 124 thereof, respectively, i.e., via the unit-to-unit coupling. In particular, FIG. 7, illustrates the coupler 114 that is associated with the first pumping unit 104 and coupling the second pumping unit 124, according to one or more embodiments. FIG. 8 illustrates the coupler 134 that is associated with the second pumping unit 124 and coupling the first pumping unit 104, according to one or more embodiments. FIG. 9 illustrates the first and second couplers 116, 226 that are associated with the first and second pumping units 104, 124 thereof, respectively, and coupling to each other, according to one or more embodiments.

With reference to FIG. 5, the first and second pumps 102, 122 couple to each other by the first pumping unit 104 and the driveline 130 of the second pump 122, i.e., via the unit-to-driveline coupling. In particular, FIG. 10 illustrates the coupler 114 that is associated with the first pumping unit 104 and coupling the driveline 130 of the second pump 122, according to one or more embodiments. FIG. 11 illustrates the coupler 134 that is associated with the driveline 130 of the second pump 122 and coupling the first pumping unit 104, according to one or more embodiments. FIG. 12 illustrates the first coupler 116 that is associated with the first pumping unit 104 and the second coupler 226 that is associated with the driveline 130 of the second pump 122, with the first and second couplers 116, 226 coupling to each other, according to one or more embodiments.

With reference to FIG. 6, the first and second pumps 102, 122 couple to each other by the drivelines 110, 130 thereof, respectively, i.e., via the driveline-to-driveline coupling. In particular, FIG. 13 illustrates the coupler 114 that is associated with the driveline 110 of the first pump 102 and coupling the driveline 130 of the second pump 122, according to one or more embodiments. FIG. 14 illustrates the coupler 134 that is associated with the driveline 130 of the second pump 122 and coupling the driveline 110 of the first pump 102, according to one or more embodiments. FIG. 15 illustrates the first coupler 116 that is associated with the driveline 110 of the first pump 102 and the second coupler 226 that is associated with the driveline 130 of the second pump 122, with the first and second couplers 116, 226 coupling to each other, according to one or more embodiments.

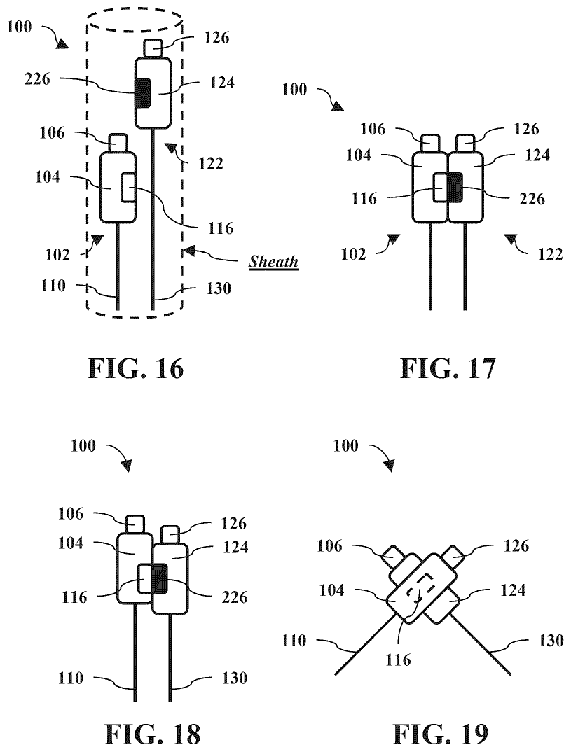

FIGS. 16-24 illustrate the assemblable pump system 100 in the unassembled state (FIGS. 16, 22-23) and the assembled state (FIGS. 17-21, 24), according to some embodiments.

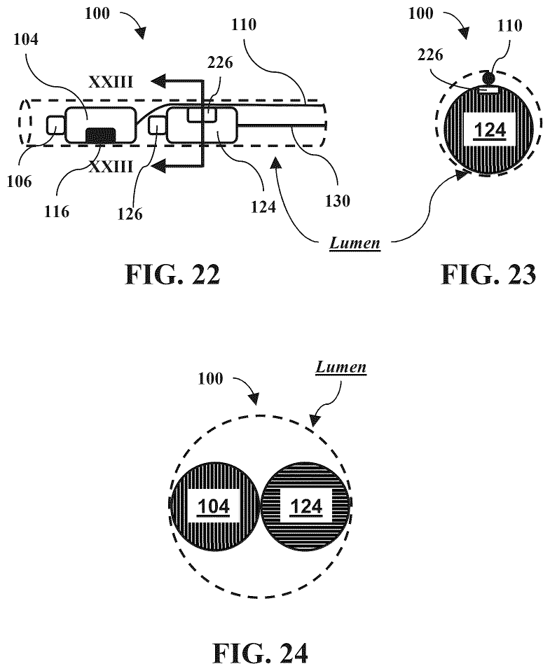

In the unassembled state, the first and second pumps 102, 122 are longitudinally positioned one after the other, as for example illustrated in the embodiments of FIGS. 16, 22, such that the cross-sectional size of the assemblable pump system 100 corresponds approximatively to the cross-sectional size of one of the first and second pumps 102, 122, when the first and second pumps 102, 122 have the same cross-sectional size. When the first and second pumps 102, 122 have a different cross-sectional sizes, the cross-sectional size of the assemblable pump system 100 in the unassembled state corresponds approximatively to the cross-sectional size of the one of the first second pumps 102, 122 having the larger cross-sectional size. The assemblable pump system 100 in the unassembled state is containable in a sheath (shown in dashed lines as an optional element in FIGS. 16, 31, 34, 37, 39, 42, 44, 46, 49), which may be used to navigate a subject's body conduit, such as a subject's vasculature including the heart chambers thereof, for implanting and explanting the assemblable pump system 100.

In the assembled state, and as further described herein in relation to FIGS. 22-24, the first and second pumps 102, 122 are transversally positioned next to each other, as for example illustrated in the embodiment of FIG. 24, such that the cross-sectional size of the assemblable pump system 100 corresponds approximatively to the cross-sectional sizes of both the first and second pumps 102, 122. Therefore, the assemblable pump system 100 has a smaller cross-sectional size in the unassembled state than in the assembled state, which is advantageous for navigating, delivering, implanting, retrieving, and explanting the assemblable pump system 100 intracorporeally and intraluminally, such as in lumens of a subject's body conduit that are not necessarily amenable to larger medical devices that cannot reduce their cross-sectional sizes. Typically, the assemblable pump system 100 is converted from the unassembled state to the assembled state in situ, such as at an implantation site inside a subject's body, and then operated for example to provide hemodynamic support.

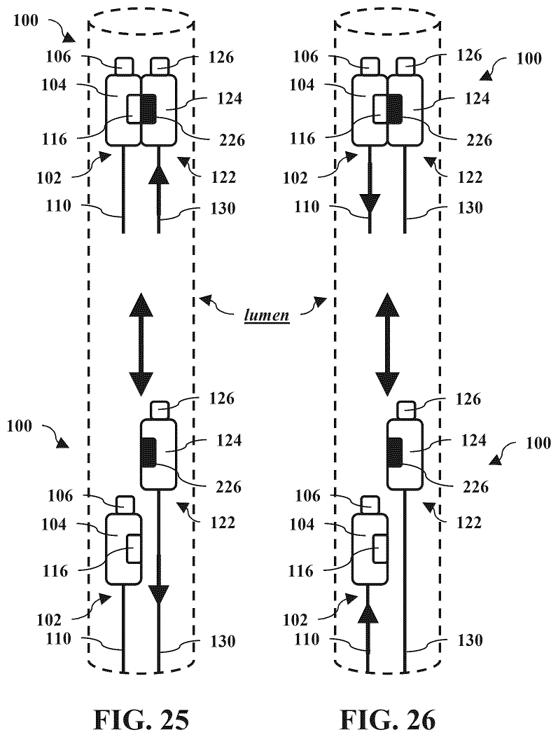

The assemblable pump system 100 may be operated in the assembled and unassembled states. In the unassembled state, as illustrated for example in FIGS. 25-26, the first and second pumping units 104, 124 are positioned offset relative to each other, similar to the offset positioning of FIG. 18. Advantageously, this relative offset position increases blood outflow performance of at least one of the first and second pumping units 104, 124.

Implantation site where the assemblable pump system 100 may be implanted includes the lumen of the vasculature, including the chambers of the heart, lumens of hollow organs, lumens of body cavities, lumens of artificially created conduit using either autologous tissue, an allograft or man-made material (e.g., a bypass made of femoral vein or of knitted polyester conduit), and any other possible lumens of a subject's body. In particular, the implantation site includes the lumens of the left and right ventricles, left and right atria, aorta, vena cava, pulmonary artery, coronary artery, coronary sinus, ductus arteriosus, renal arteries, femoral arteries, and coronary artery bypass graft.

FIG. 16 illustrates the assemblable pump system 100 in the unassembled state with the first and second pumping units 104, 124, which in this case are impeller pumps, longitudinally positioned one after the other, according to one or more embodiments. In particular, when contained in a sheath, the first and second pumping units 104, 124 are disposed head-to-tail relative to each other, i.e., that the distal, impeller-receiving end portion of the first pumping unit 104 is oriented toward the proximal, driveline-receiving end portion of the second pumping unit 124. The first pumping unit 104 is positioned proximally relative to the second pumping unit 124 such that the second pumping unit 124 exits first from the sheath, followed by the first pumping unit 104.

Although usually desirable, a sheath is not necessarily needed for navigating, delivering, implanting, retrieving, and explanting the assemblable pump system 100 in vivo. Indeed, each of the first and second pumps 102, 122 may be navigated, delivered, implanted, retrieved, and explanted in vivo simply by pulling and/or pushing of the drivelines 110, 130, respectively, without the need for the first and second pumps 102, 122 to be contained in a sheath.

Each of the first and second pumps 102, 122 may also define a railing guide hole or similar structure that is configured to receive a guide wire (not shown) therethrough for delivering and implanting the first and second pumps 102, 122 in vivo by railing them over the guide wire. Briefly, the guide wire is first routed along a lumen of a subject's body conduit, such as the subject's vasculature, up to an implantation site. Then the first and second pumping units 104, 124 in the unassembled state and longitudinally positioned one after the other are railed over the guide wire and thus guided to the implantation site where they may be assembled and operated.

FIGS. 17-21 illustrate the assemblable pump system 100 in the assembled state with the first and second pumping units 104, 124, which in this case are also impeller pumps, transversally positioned next to each other via the unit-to-unit coupling, according to some embodiments. Depending on the coupling modes and the integration of the couplers 114, 134, 116, 226 to the first and second pumps 102, 122, the first and second pumping units 104, 124 may have various positions relative to each other.

As illustrated in FIG. 17, the first and second pumping units 104, 124 in the assembled state are parallelly positioned relative to each other, according to one or more embodiments. In particular, the distal, impeller-receiving end portions of the first and second pumping units 104, 124 are positioned side by side, and the proximal, driveline-receiving end portions of the first and second pumping units 104, 124 are also positioned side by side.

As illustrated in FIG. 18, the first and second pumping units 104, 124 in the assembled state are parallelly positioned offset relative to each other, according to one or more embodiments. In particular, the distal, impeller-receiving end portions of the first and second pumping units 104, 124 are longitudinally positioned offset relative to each other, and the proximal, driveline-receiving end portions of the first and second pumping units 104, 124 are also longitudinally positioned offset relative to each other. The couplers 114, 134, 116, 226, as the case may be, may be integrated to the first and second pumping units 104, 124 in an offset manner to achieve the relative parallel offset position of the first and second pumping units 104, 124 in the assembled state. Advantageously, this relative offset position increases blood outflow performance of at least one of the first and second pumping units 104, 124.

As illustrated in FIG. 19, the first and second pumping units 104, 124 in the assembled state intersect each other (only the first coupler 116 is shown in dashed lines as a way to see it through the first pumping unit 104 in FIG. 19), according to one or more embodiments. In particular, the distal, impeller-receiving end portion and the driveline-receiving end portion of the first pumping unit 104 are generally positioned toward opposite sides of the second pumping unit 124. Similarly, the distal, impeller-receiving end portion and the driveline-receiving end portion of the second pumping unit 124 are generally positioned toward opposite sides of the first pumping unit 104. The couplers 114, 134, 116, 226, as the case may be, may be integrated to a corresponding intermediate portion of the first and second pumping units 104, between the distal, impeller-receiving end portions and the driveline-receiving end portions thereof, to achieve this intersecting position.

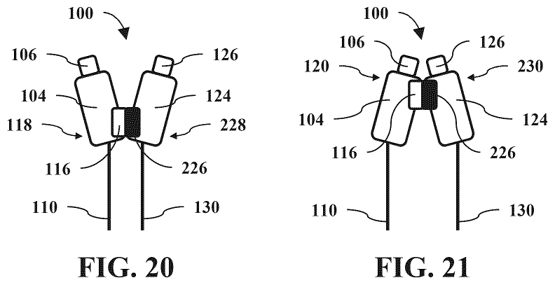

As illustrated in FIG. 20, the first and second pumping units 104, 124 in the assembled state are non-parallelly positioned at an angle relative to each other, according to one or more embodiments. In particular, the distal, impeller-receiving end portions of the first and second pumping unit 104, 124 are further spaced apart than the proximal, driveline-receiving end portions of the first and second pumping units 104, 124. As such, the proximal, driveline-receiving end portions of the first and second pumping units 104, 124 converge toward each other. The couplers 114, 134, 116, 226, as the case may be, may be integrated to the proximal, driveline-receiving end portions of the first and second pumping units 104, 124 to achieve this non-parallel and angular position of the first and second pumping units 104, 124 in the assembled state.

As illustrated in FIG. 21, the first and second pumping units 104, 124 in the assembled state are also non-parallelly positioned at an angle relative to each other, according to one or more embodiments. In particular, the proximal, driveline-receiving end portions of the first and second pumping units 104, 124 are further spaced apart than the distal, impeller-receiving end portions of the first and second pumping unit 104, 124. As such, the distal, impeller-receiving end portions of the first and second pumping units 104, 124 converge toward each other. The couplers 114, 134, 116, 226, as the case may be, may be integrated to the distal, impeller-receiving end portions of the first and second pumping unit 104, 124 to achieve this non-parallel and angular position of the first and second pumping units 104, 124 in the assembled state.

Although FIGS. 16-21 illustrate the first and second couplers 116, 226, the couplers 114,134 may achieve the same relative positioning between the first and second pumping units 104, 124 in the assembled state as illustrated in FIGS. 17-21 and described herein, with the necessary change(s), appreciable to the skilled addressee, having been made, if applicable.

Being convertible between the unassembled and assembled states, the assemblable pump system 100 can advantageously have its cross-sectional size changed to enable a reduction of its insertion profile for inserting the assemblable pump system 100 through a subject's intracorporeal body access, such as a subject's intraluminal body access, and to enable a reduction of its footprint for navigating the assemblable pump system 100 into lumens of a subject's body conduit, such as the subject's vasculature including the heart chambers thereof. This makes the assemblable pump system 100 amenable to subject's body conduits that are generally not possible for larger medical devices that cannot reduce their cross-sectional sizes.

Indeed, as illustrated in the embodiments of FIGS. 22-23, the assemblable pump system 100 in the unassembled state with the first and second pumping units 104, 124 longitudinally positioned one after the other into a lumen of a subject's body conduit (as shown in FIG. 22; the subject's body conduit is shown in dashed lines as an environmental element in FIGS. 22-24) has a cross-sectional size corresponding to the cross-sectional size of the first pumping unit 104 plus the cross sectional of the driveline 130 of the second pump 122 (as shown in FIG. 23).

For comparison purpose, as illustrated in the embodiment of FIG. 24, the assemblable pump system 100 in the assembled state with the first and second pumping units 104, 124 transversally positioned next to each other into a lumen of a subject's body conduit has a maximal cross-sectional size corresponding to the cross-sectional sizes of both the first and second pumping units 104, 124. Therefore, the assemblable pump system 100 in the unassembled state can be advantageously inserted through a subject's intracorporeal body access and navigated in lumens of a subject's body conduits where the assemblable pump system 100 in the assembled state-or any other medical devices so sized-cannot be due to size limitation.

Referring back to FIG. 23, in some embodiments where the driveline 130 transmits electricity to the second pumping unit 124, which provides electricity to the first pumping unit 104 through the couplers 114, 134, 116, 226, as the case may be, the driveline 110 may be void of the electrical conductor 112 and thus be smaller in cross-sectional size, as compared to when provided with the electrical conductor 112. This in turn advantageously reduces the cross-sectional size of the assemblable pump system 100 in the unassembled state.

FIGS. 25-26 illustrate the conversion of the assemblable pump system 100 from the unassembled state to the assembled state and from assembled state to the unassembled state by manipulation of at least one of the drivelines 110, 130 to correspondingly move at least one of the first and second pumps 102, 122 along a lumen of a subject's body conduit (which is shown in dashed lines as an environmental object in FIGS. 25-26). In particular, manipulation of the driveline 130 and/or the driveline 110 (when present) correspondingly moves at least one of the first and second pumps 102, 122 toward the other one of the first and second pumps 102, 122 and causes the first coupler 116 to physically engage and couple the second coupler 226. Manipulation of the driveline 130 and/or the driveline 110 (when present) correspondingly moves at least one of the first and second pumps 102, 122 away from the other one of the first and second pumps 102, 122 and causes the first coupler 116 to physically disengage and uncouple the second coupler 226.

As illustrated, the first pumping unit 104 is positioned proximally relative to the second pumping unit 124 in a lumen of a subject's body conduit and ready for assembling, according to one or more embodiments. For conversion from the unassembled state to the assembled state, the driveline 130 of the second pump 122 may be manipulated and pulled (as indicated by the arrow pointing downwardly in FIG. 25) so as to move the second pumping unit 124 toward the first pumping unit 104 along the lumen for assembling. Alternatively or additionally, the driveline 110 of the first pump 102 (when present) may be manipulated and pushed (as indicated by the arrow pointing upwardly in FIG. 26) so as to move the first pumping unit 104 toward the second pumping unit 124 along the lumen for assembling. For conversion from assembled state to the unassembled state, the driveline 130 of the second pump 122 may be manipulated and pushed (as indicated by the arrow pointing upwardly in FIG. 25) so as to move the second pumping unit 124 away from the first pumping unit 104 along the lumen for unassembling. Alternatively or additionally, the driveline 110 of the first pump 102 (when present) may be manipulated and pulled (as indicated by the arrow pointing downwardly in FIG. 26) so as to longitudinally move the first pumping unit 104 away from the second pumping unit 124 along the lumen for unassembling.

The drivelines 110, 130 are configured to be navigable in a subject's body, such as in lumens of subject's body conduits. As such, the drivelines 110, 130 may be flexible enough to be appropriately introduced and navigated intracorporeally and intraluminally. The drivelines 110, 130 are also configured to be manipulated, such as by pulling and/or pushing, for converting the assemblable pump system 100 between the unassembled and assembled states, as described herein. In particular, a respective portion of each of the drivelines 110, 130 may be disposed intraluminally, intracorporeally, or extracorporeally and may be manipulated for converting the assemblable pump system 100 in a lumen of a subject's body conduit from the unassembled state to the assembled state and from the assembled state to the unassembled state.

To facilitate pushing the pumping units 104, 124, each of the drivelines 110, 130 may be separately provided with a longitudinal cavity that is configured to receive a guide wire therein. Guide wires of various stiffnesses or structural rigidities may be inserted in the longitudinal cavity in order to modulate the stiffness or structural rigidity of the drivelines 110, 130, mitigating or preventing the bending and folding of the drivelines 110, 130 upon applying a pushing force thereto. For example, referring to FIG. 25, a guide wire of a given stiffness or structural rigidity may be inserted in the driveline 110 (when present) in order to maintain the first pumping unit 104 in place by applying a pushing force to the driveline 110, while the driveline 130 of the second pump 122 is pulled (as indicated by the arrow pointing downwardly in FIG. 25) for assembling. Also, a guide wire of a given stiffness or structural rigidity may be inserted in the driveline 130 so that a pushing force may be appropriately applied thereto without bending or folding it in order to push the second pumping unit 124 away from the first pumping unit 104 (as indicated by the arrow pointing upwardly in FIG. 25 for unassembling.

The use of guide wires of various stiffnesses or structural rigidities can be useful for implanting and explanting the assemblable pump system without using a sheath (i.e. via a non-transcatheter approach) and for providing tactile feedback to an operator as the first and second pumps 102, 122 are navigated intraluminally. Selection of such guide wires and stiffness or structural rigidity thereof may depend on a subject's vasculature morphology and/or the medical procedure being performed.

In one or more embodiments, the first and second pumps 102, 122 are configured to be assembled together and unassembled from each other by stereotaxis, such as by the use of a stereotaxis magnetic navigation system. In this case, the assemblable pump system 100 may not be provided with the drivelines 110, 130, and may be configured to receive power wirelessly, such as by transcutaneous energy transfer (TET), by a receiver coil for operation of the same.

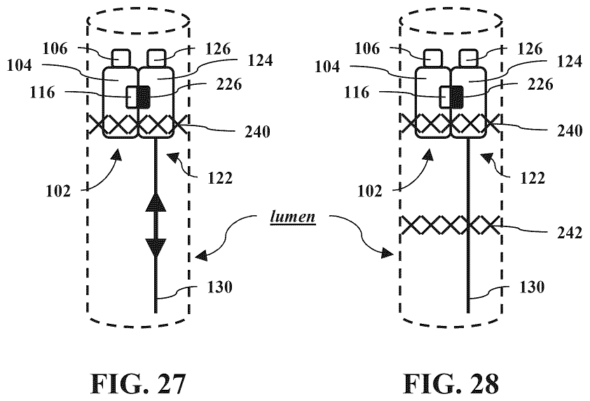

FIG. 27 illustrates the assemblage pump system 100 provided with a pumping unit anchor 240 removably or non-removably attached to the first pumping unit 104, according to one or more embodiments. The pumping unit anchor 240 has an unexpanded state that is configured for navigating, delivering, and implanting the assemblage pump system 100 in vivo, and an expanded state that is configured to anchor the assemblage pump system 100 in vivo. As illustrated, the assemblage pump system 100 in the assembled state is anchored in a lumen of a subject's body conduit (which is shown in dashed lines as an environmental object) by the pumping unit anchor 240 in the expanded state that engages an intraluminal wall of the subject's body conduit. So anchored, the first pumping unit 104 is immobilized in the lumen and the driveline 130 of the second pump 122 can be manipulated to move the second pumping unit 124 toward the first pumping unit 104 for assembling. Alternatively or additionally, the first pumping unit 104 may be immobilized in the lumen by holding the driveline 110 (when present) that receives a guide wire of appropriate stiffnesses or structural rigidities in the longitudinal cavity thereof, as described herein.

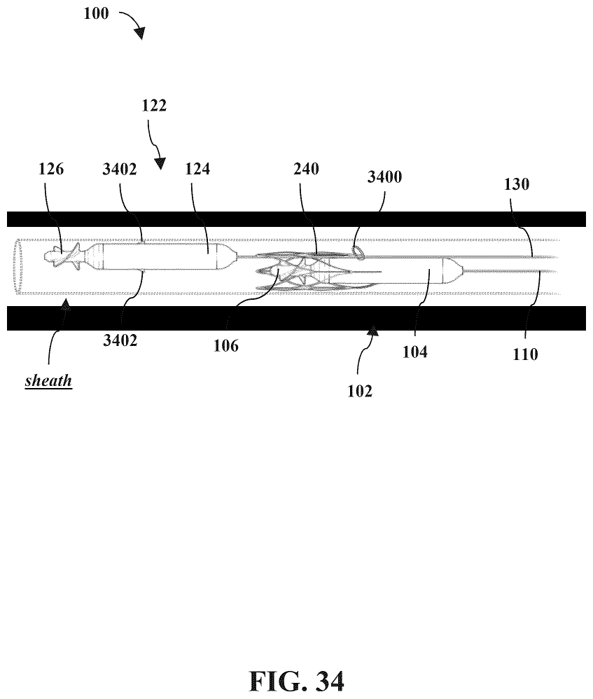

FIG. 28 illustrates the assemblage pump system 100 provided with a pumping unit anchor 240 removably or non-removably attached to the first pumping unit 104, and also provided with a driveline anchor 242 removably or non-removably attached to the first pumping unit 104, according to one or more embodiments. The driveline anchor 242 being generally similar to the pumping unit anchor 240, the driveline anchor 242 will not be further described herein for the sake of brevity. It will be appreciated that a similar description applies to the driveline anchor 242 and the pumping unit anchor 240, including the description thereof in relation to FIGS. 34-36, with the necessary change(s), appreciable to the skilled addressee, having been made, if applicable.

Advantageously, the combination of the pumping unit and driveline anchors 240, 242 provides more anchoring stability to the assemblage pump system 100 when implanted intraluminally, as compared when one of the pumping unit and driveline anchors 240, 242 is used alone. Because of the longitudinal distance separating the pumping unit and driveline anchors 240, 242 along the first pump 102, the assemblage pump system 100 in the assembled state is less prone to pivot and contact the intraluminal wall of a subject's body conduit in response to a movement of the driveline 130 and the driveline 110 (when present).

In some embodiments, the assemblage pump system 100 may be provided with only the driveline anchor 242 removably or non-removably attached to at least one of the drivelines 110, 130, and no pumping unit anchor 240 is provided to the assemblable pump system 100.

The pumping unit and driveline anchors 240, 242 may be self-expandable anchors that are biased toward the expanded state and, as such, may be converted from the expanded state to the unexpanded state by forcing them in a sheath.

Although FIGS. 25-28 illustrate the first and second couplers 116, 226, the first and second pumping units 104, 124 may be provided with a corresponding one of the couplers 114, 134, and the driveline 130 and/or the driveline 110 (when present) may be manipulated to correspondingly move at least one of the first and second pumps 102, 122, as the case may be, for converting the assemblable pump system 100 between the unassembled and assembled states, with the necessary change(s), appreciable to the skilled addressee, having been made, if applicable.

In particular, manipulation of the driveline 130 and/or the driveline 110 (when present) moves at least one of the first and second pumps 102, 122 toward the other one of the first and second pumps 102, 122 and causes the coupler 114 to physically engage and couple the second pump 122 or causes the coupler 134 to physically engage and couple the first pump 102. Manipulation of the driveline 130 and/or the driveline 110 (when present) moves at least one of the first and second pumps 102, 122 away from the other one of the first and second pumps 102, 122 and causes the coupler 114 to physically disengage and uncouple the second pump 122 or causes the coupler 134 to physically disengage and uncouple the first pump 102.

Further, although FIGS. 25-26 illustrate a unit-to-unit coupling between the first and second pumping units 104, 124, the driveline 130 and/or the driveline 110 (when present) may be manipulated to correspondingly move at least one of the first and second pumps 102, 122, as the case may be, for converting the assemblable pump system 100 between the unassembled and assembled states via a unit-to-driveline coupling and a driveline-to-driveline coupling, with the necessary change(s), appreciable to the skilled addressee, having been made, if applicable.

Referring back to FIGS. 1-3, the assemblable pump system 100 may be powered in multiple ways. The assemblable pump system 100 may be electrically powered and, in this case, the drivelines 130 and the driveline 110 (when present) may be referred to as electrical cables or electrical wires, according to one or more embodiments. In particular, the assemblable pump system 100 may be powered by the driveline 130 alone that transmits power from a power source to the second pumping unit 124, which further provides power to the first pumping unit 104 through the couplers 114, 134, 116, 226, as the case may be. In this case, each of the couplers 114, 134, 116, 226 have a physical component configured to physically engage and couple the first and second pumps 102, 122 to each other, and an electrical component configured to transmit power between the first and second pumping units 104, 124 when the first and second pumps 102, 122 are in the assembled state. Alternatively, the assemblable pump system 100 may be powered by both the driveline 130 and the driveline 110 (when provided). In this case, the first and second pumps 102, 122 may be operated independently from each other. The couplers 114, 134, 116, 226 may or may not have the electrical component.

The assemblable pump system 100 may be powered by transcutaneous energy transfer (TET), according to one or more embodiments. In particular, one of the first and second pumps 102, 122 may be configured to be powered by TET and therefore to wirelessly receive power from a power source that is disposed extracorporeally. The one of the first and second pumps 102, 122 that is powered by TET provides power to the other one of the first and second pumps 102, 122 through the couplers 114, 134, 116, 226, as the case may be and as described herein for the physical and electrical components thereof. Alternatively, both the first and second pumps 102, 122 may be configured to be powered by TET. In this case, the first and second pumps 102, 122 may be operated independently from each other. The couplers 114, 134, 116, 226 may or may not have the electrical component.

The assemblable pump system 100 may be mechanically powered, according to one or more embodiments. In particular, each of the driveline 130 and the driveline 110 (when present) may include a respective driveshaft that is rotatably coupled to a respective one of the first and second impellers 106, 126. In this case, the first and second drive units 108,128 are not integrated to the first and second pumping units 104, 124 and are instead integrated to a distal end portion of the corresponding drivelines 110, 130 and driveshaft thereof that projects away from the first and second pumping units 104, 124, respectively.

The driveline 110 (when present) and the driveline 130 may or may not be detachable from the first and second pumping units 104, 124, respectively. For example, an assemblable pump system 100 that is powered through both drivelines 110, 130 may have one of the drivelines 110, 130 detached, such that the assemblable pump system 100 may be operated through only one of the drivelines 110, 130.

The assemblable pump system 100 is not limited to the first and second pumps 102, 122 and may include any number of pumps that may be converted from an unassembled state to an assembled state and from the assembled state to the unassembled state by manipulation of one or more corresponding drivelines, with the necessary change(s), appreciable to the skilled addressee, having been made, if applicable.

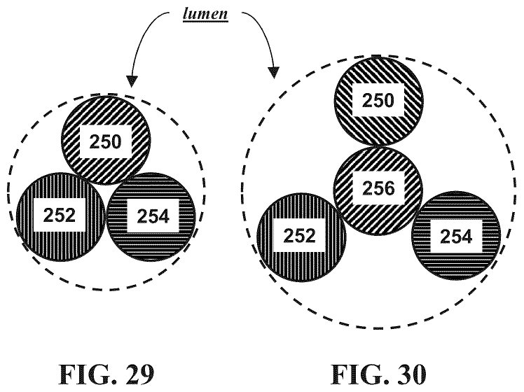

FIG. 29 illustrates the assemblable pump system 100 that includes three pumps 250, 252, 254. The pumps 250, 252, 254 may be converted between an unassembled state and an assembled state by manipulation of at least one of the drivelines thereof. As illustrated, the pumps 250, 252, 254 are in the assembled configuration in a lumen of a subject's body conduit (which is shown in dashed lines as an environmental object in FIG. 29) and are transversally positioned next to each other to define generally a triangle, according to one or more embodiments.

FIG. 30 illustrates the assemblable pump system 100 that includes four pumps 250, 252, 254, 256. The pumps 250, 252, 254, 256 may be converted between an unassembled state and an assembled state by manipulation of at least one of the drivelines thereof. As illustrated, the pumps 250, 252, 254, 256 are in the assembled configuration in a lumen of a subject's body conduit (which is shown in dashed lines as an environmental object in FIG. 29) and are transversally positioned next to each other to define generally a triangle receiving one of the pump 256 at the center point thereof, according to one or more embodiments.

The assemblable pump system 100 may include up to ten pumps having a cross-sectional size of about between 4-6 mm and still be able, when in the unassembled state, to be navigated to and delivered in a lumen of a subject's descending aorta. Then, the assemblable pump system 100 may be converted from the unassembled state to the assembled state in situ and be operated for producing a hemodynamic support to the subject.

Referring for example to FIGS. 29-30, depending on the number and cross-sectional size of the pumps, the assemblable pump system 100 in the assembled state may not fully occupy the full diameter space of the lumen and thus may allow blood to flow between the pumps and the lumen wall and/or between pumps themselves. Advantageously, blood may continue to flow downstream the assemblable pump system 100 in the unlikely event of device failure or malfunction.

Advantageously, given its multi-pumps design, the assemblable pump system 100 may be operated at a relatively low impeller rotational speed, such as about between 5,000 rpm and 30,000 rpm for each one of the first and second impellers 106, 126, and still be able to generate a blood outflow appropriate for providing hemodynamic support, such as about between 1 L/min and 3 / min. For example, an assemblable pump system 100 provided with three pumps that are operated at about between 5,000 rpm and 30,000 rpm would generate about between 1.5 L/min and 5 L/min. By way of comparison, some single impeller blood pumps generally required to be operated at a relatively higher impeller rotational speed, such as about between 15,000 and 60,000rpm, in order to generate a similar blood outflow appropriate for providing hemodynamic support.

On balance, operation of some single impeller blood pump at a relatively high impeller rotational speed generates more blood damage, such as blood damage caused by impeller-induced scalar shear stress, as compared to the operation of a multi-pumps design like the assemblable pump system 100 at a relatively low impeller rotational speed. Accordingly, the assemblable pump system 100 may be implanted and operated in a subject's vasculature for a relatively longer period of time than some single impeller blood pump and, as such, the assemblable pump system 100 is comparatively more suitable for long-term or chronic medical condition, such as bridge-to-transplant or destination therapy. As compared to single pump device, the multi-pump design of the assemblable pump system 100 also advantageously confers more pump redundancy in the event of device failure.

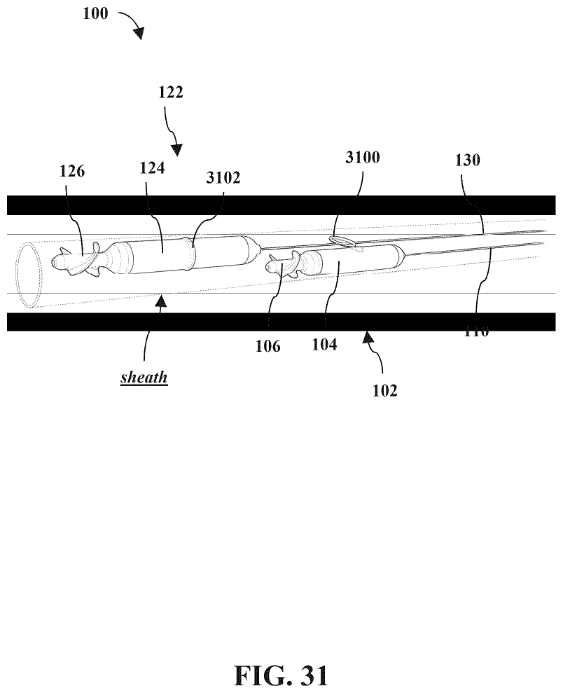

With reference now to FIGS. 31-50, some embodiments of the assemblable pump system 100 will be described.

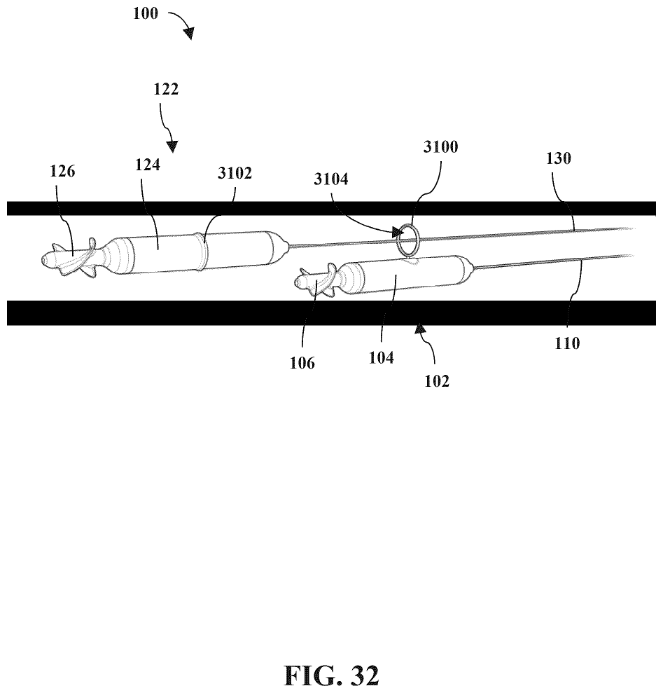

FIGS. 31-33 illustrate the assemblable pump system 100 including the first pump 102 provided with the coupler 114 that is a pump-receiving coupler 3100, and the second pump 122 provided with an abutment stop, such as a bulge 3102, according to one or more embodiments. The pump-receiving coupler 3100 defines an opening 3104 (best shown in FIG. 32) that is sized and shaped to slidably receive the driveline 130 of the second pump 122 and the second pump 122 therein. The pump-receiving coupler 3100 is configured to abut the bulge 3102 in order to stop the relative sliding of the first and second pumps 102, 122 upon actuation. Also, the pump-receiving coupler 3100 may be configured to be convertible between an undeployed state and a deployed state, as described hereinafter.

As illustrated in FIG. 31, the assemblable pump system 100 is in the unassembled state with the first and second pumps 102, 122 contained in a sheath (which is shown in dashed lines as an environmental object), and the pump-receiving coupler 3100 in an undeployed state for delivery to an implantation site. While not illustrated as such, the pump-receiving coupler 3100 may be shaped and sized, when in the undeployed conformation, to conform the outer size and outer shape of the first pumping unit 104 so that the cross-sectional size of the first pumping unit 104 is the same or substantially the same as if there would be no pump-receiving coupler 3100 provided to the first pump 102.

As illustrated in FIG. 32, the assemblable pump system 100 is still in the unassembled state, but with the first and second pumps 102, 122 having been exited from the sheath (not shown) and delivered at the implantation site, ready to be assembled together by manipulation of at least one of the drivelines 110, 130, respectively, as described herein. Further, the pump-receiving coupler 3100 is now in the deployed state, having converted from the undeployed state. The pump-receiving coupler 3100 may convert from the undeployed state to the deployed state by itself when made of a memory material, such as nitinol or silicone, and when overcomeably biased toward the deployed state. Alternatively, the pump-receiving coupler 3100 may convert from the undeployed state to the deployed states by being overcomeably spring-loaded toward the deployed state, such as by pivoting or by translating relative to the first pumping unit 104. When spring-loaded, the pump-receiving coupler 3100 may or may not be made of a memory material.

In embodiments where the assemblable pump system 100 is transcatheterly deliverable, the pump-receiving coupler 3100, when overcomeably biased toward the deployed state, is configured to auto-convert from the undeployed state to the deployed state as the sheath releases the constraint imposed on the pump-receiving coupler 3100 while being exited therefrom during delivery. Conversely, the pump-receiving coupler 3100 may also be configured to convert back from the deployed state to the undeployed state as the assemblable pump system 100 is re-sheated for retrieval.

Alternatively, the driveline 110 of the first pump 102 may include an actuation mechanism (not shown) coupled to the pump-receiving coupler 3100 that is operable by an operator in such a way to convert the pump-receiving coupler 3100 between the undeployed and deployed states, when the assemblable pump system 100 is outside an optional sheath.

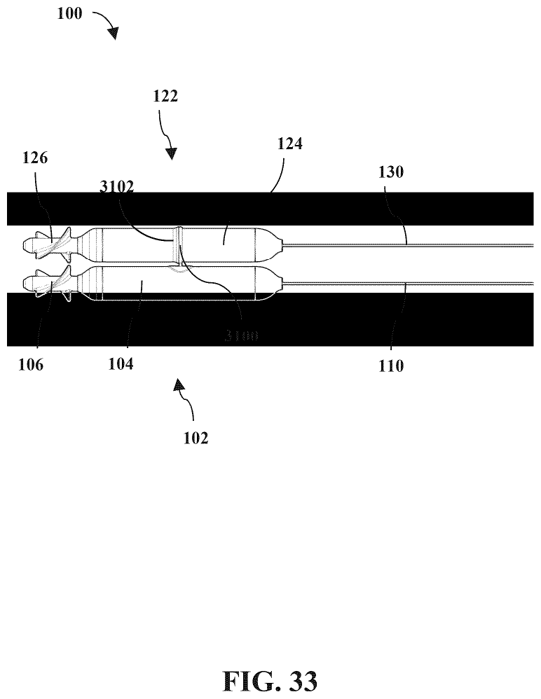

As illustrated in FIG. 33, the assemblable pump system 100 is now in the assembled state, as a result of the slidable actuation of at least one of the drivelines 110, 130 and relative movement of at least one of the first and second pumps 102, 122, as described herein. The second pumping unit 124 is received in the opening 3104 of the pump-receiving coupler 3100, which is attached to the first pumping unit 104. The bulge 3102 of the second pump 122 abuts the pump-receiving coupler 3100, preventing relative movement of the first and second pumping units 104, 124 by pushing the driveline 110 of the first pump 102 and/or pulling the driveline of the second pump 122.

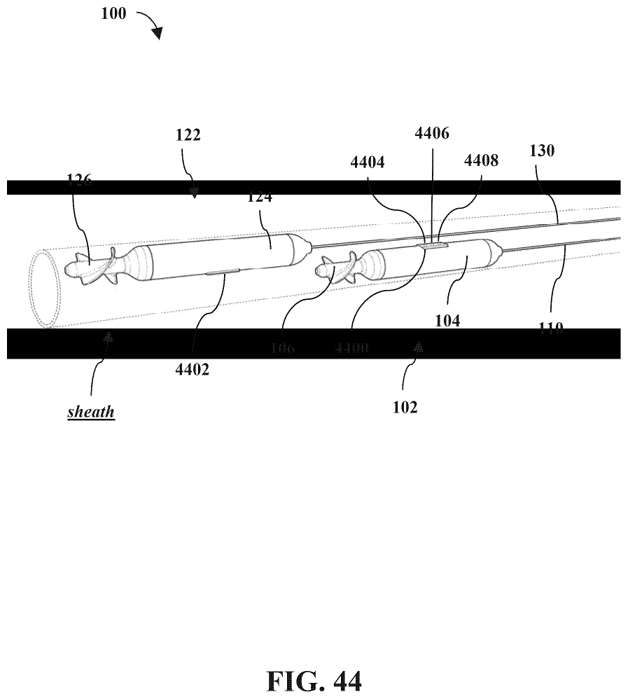

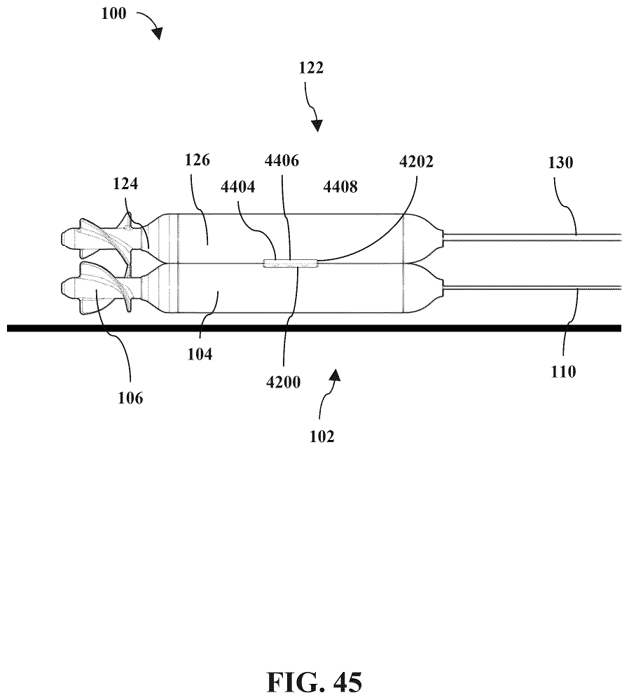

In embodiments, the assemblable pump system 100 may be provided with the pump-receiving coupler 3100 in addition to be also provided with one or more magnet(s) (not shown), and/or the first and second electrical connectors 4400, 4402 (as described with reference to FIGS. 44-45; not shown in FIGS. 31-33). The magnet(s) and/or the first and second electrical connectors 4400, 4402 may be provided to the pump-receiving coupler 3100, the bulge 3102, or be provided as separate component(s) to the first and second pumps 102, 122.

For example, a magnet (not shown) may be provided to one of the pump-receiving coupler 3100 and bulge 3102, while the other one of the pump-receiving coupler 3100 and bulge 3102 may include a ferromagnetic material (not shown) or another magnet (not shown) for magnetically coupling the magnet. Alternatively, the two magnets and the ferromagnetic material may be provided elsewhere to the first and second pumps 102, 122.

Still for example, the first and second electrical connectors 4400, 4402 may be provided to the pump-receiving coupler 3100 and bulge 3102, respectively, for transmitting electricity between them when electrically connected together, as described hereinafter. Alternatively, the first and second electrical connectors may be provided elsewhere to the first and second pumps 102, 122.

Whereas in this embodiment the pump-receiving coupler 3100 is disclosed as a ring that is sized and shaped to receive a tubular-sized pumping unit therein, it will be appreciated that the pump-receiving coupler 3100 and the second pumping unit 124 may each have any other complementary mating size and shape allowing the first and second pumps 102, 122 to inter-engage and assemble together.

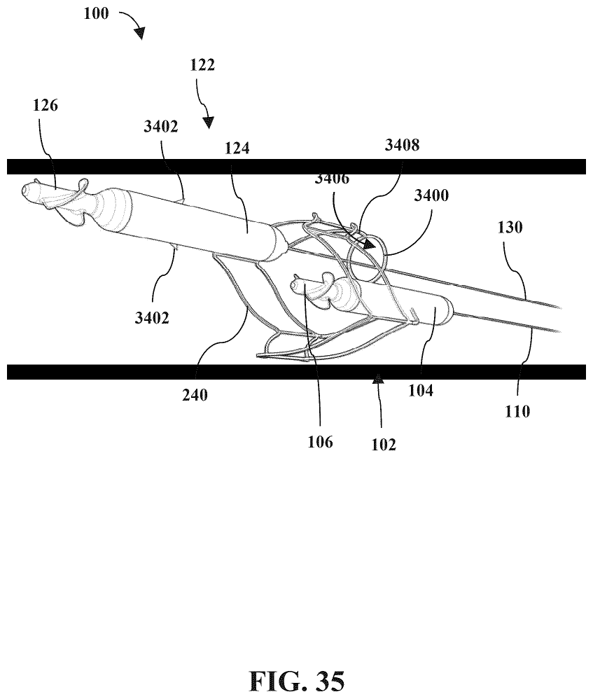

FIGS. 34-36 illustrate the assemblable pump system 100 including the first pump 102 provided with the pumping unit anchor 240 having the coupler 114 that is a pump-receiving coupler 3400, and the second pump 122 provided with an abutment stop, such a pair of tabs 3402, according to one or more embodiments. Similar to the pump-receiving coupler 3100, the pump-receiving coupler 3400 defines an opening 3406 (best shown in FIG. 35) that is sized and shaped to slidably receive the driveline 130 of the second pump 122 and the second pump 122 therein. The pump-receiving coupler 3400 is configured to abut the pair of tabs 3402 (which in this embodiment replaces the bulge 3102) in order to stop the relative sliding of the first and second pumps 102, 122 upon actuation.

The pump-receiving coupler 3400 being generally similar to the pump-receiving coupler 3100, the pump-receiving coupler 3400 will be briefly described hereinafter. It will be appreciated that a similar description applies to the pump-receiving couplers 3400 and 3100, with the necessary change(s), appreciable to the skilled addressee, having been made, if applicable.

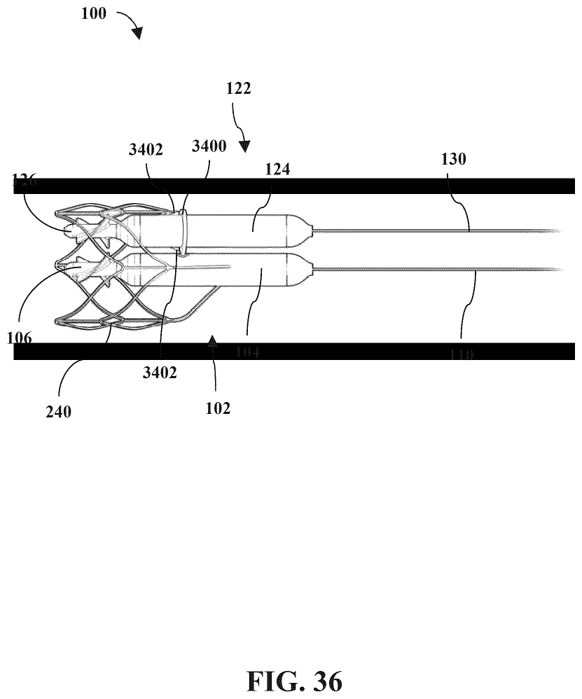

The pumping unit anchor 240 has an unexpanded state, as illustrated in FIG. 34, and an expanded state, as illustrated in FIG. 35-36. In the unexpanded state, the pumping unit anchor 240 is sized and shaped for delivering the assemblable pump system 100 in a lumen. For transcatheter delivery, the assemblage pump system 100 in the unassembled state with the pumping unit anchor 240 in the unexpanded state is contained in a sheath (which is shown in dashed lines as an environmental object in FIG. 34). In this case, the pumping unit anchor 240 may be made of a memory material, such as nitinol, and may be overcomeably biased toward the expanded state so as to be self-expandable. When biased toward the expanded state, the pumping unit anchor 240 is maintained in the unexpanded state by being constrained by the sheath and auto-convert to the expanded state while being exited from the sheath as constraint is removed. Alternatively, the pumping unit anchor 240 may be balloon expandable. In this case, the pumping unit anchor 240 may be made of a malleable material, such as stainless steel or titanium.

In the expanded state, the pumping unit anchor 240 is sized and shaped to engage and apply a force to a wall of a lumen into which the assemblage pump system 100 is implanted. Anchored to lumen wall, the assemblage pump system 100 is maintain in place so that at least one of the first and second pumping units 104, 124 is immobilized, while the other one of the first and second pumping units 104, 124 may be moved, such as by pulling or pushing on a corresponding one of the drivelines 110, 130, to assemble and unassemble the assemblable pump system 100.

As illustrated in FIG. 34, the assemblable pump system 100 is in the unassembled state with the first and second pumps 102, 122 contained in a sheath, the pumping unit anchor 240 in the unexpanded state, and the pump-receiving coupler 3400 in an undeployed state for delivery to an implantation site.

As illustrated in FIG. 35, the assemblable pump system 100 is still in the unassembled state. However, the first and second pumps 102, 122 have been exited from the sheath (not shown) and delivered at the implantation site, the pumping unit anchor 240 is now in the expanded state for anchoring the assemblage pump system 100 at the implantation site, and the pump-receiving coupler 3400 is now in a deployed state. The first and second pumps 102, 122 are ready to be assembled together by manipulation of at least one of the drivelines 110, 130, respectively, as described herein. As illustrated, the pump-receiving coupler 3400 is integrated to an arm 3406 of the pumping unit anchor 240. As a consequence, conversion of the pumping unit anchor 240 between the unexpanded and expanded states converts also the pump-receiving coupler 3400 between the undeployed and deployed states, respectively. The pumping unit anchor 240 may auto-convert from the unexpanded state to the expanded state when made of a memory material, such as nitinol, that is overcomeably biased toward the expanded state. Alternatively, the pumping unit anchor 240 may be made of a malleable material and be converted from the unexpanded state to the expanded state by being expended with a balloon catheter, as known in the art.

As illustrated in FIG. 36, the assemblable pump system 100 is now in the assembled state, as a result of the slidable actuation of the driveline 130 of the second pump 122 and movement thereof relative to the first pump 102 that is anchored and immobilized, as described herein. The second pumping unit 124 is received in the opening 3406 of the pump-receiving coupler 3400, which is integrated to the pumping unit anchor 240. The pair of tabs 3402 abuts the pump-receiving coupler 3400, preventing relative movement of the first and second pumping units 104, 124 by pulling the driveline 130 of the second pump 122.

In embodiments, similar to the pump-receiving coupler 3100, the assemblable pump system 100 may be provided with the pump-receiving coupler 3400 in addition to be also provided with one or more magnet(s) (not shown), and/or the first and second electrical connectors 4400, 4402 (as described in relation to FIGS. 44-45; not shown in FIGS. 34-36). It will be appreciated that a similar description applies to the pump-receiving coupler 3400 and the pump-receiving coupler 3100, with the necessary change(s), appreciable to the skilled addressee, having been made, if applicable.

Whereas in this embodiment the pump-receiving coupler 3400 is disclosed herein as being integrated to the arm 3408 of the pumping unit anchor 240, it will be appreciated that the pump-receiving coupler 3400 may in fact be integrated elsewhere on the pumping unit anchor 240, such as on a portion thereof engaging and applying a force to a lumen, so long as the second pump 122 may be coupled thereto. It will further be appreciated that the driveline 110 of the first pump 102 is optional since the pumping unit anchor 240 may be sufficient to maintain in place or immobilized the first pump 102 in a lumen so that the second pump 122 may be slidably moved relative to the anchored first pump 102 for assembling and unassembling the assemblable pump system 100.

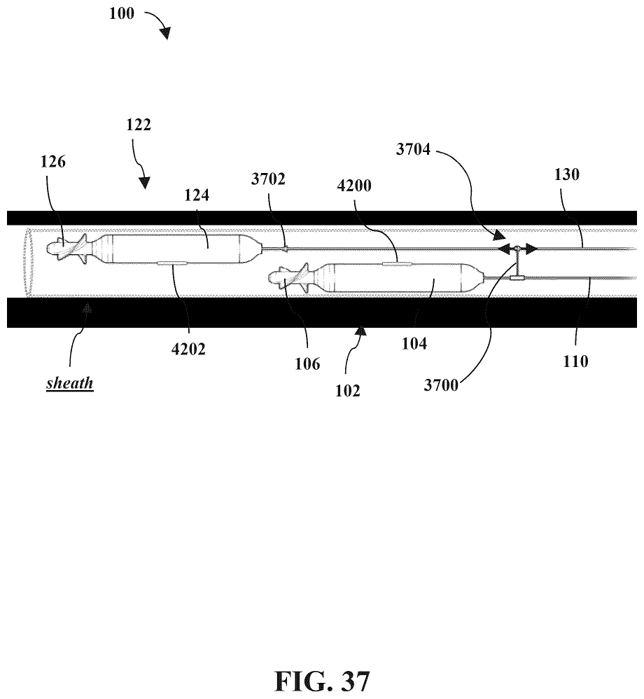

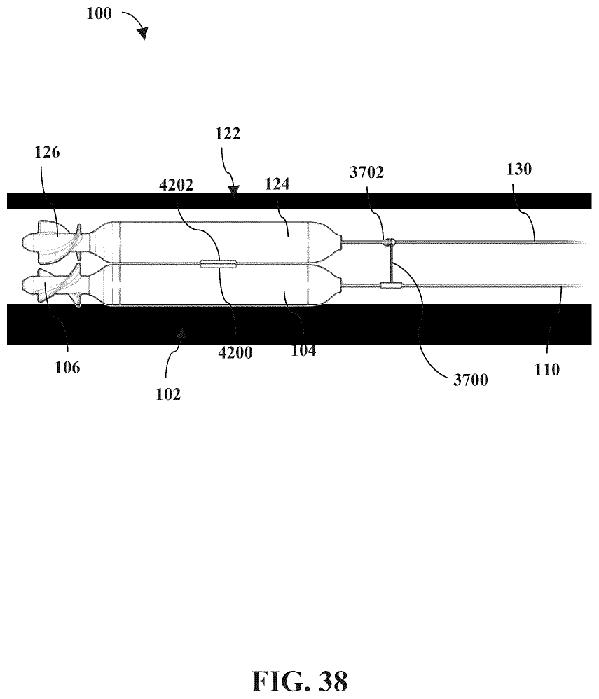

FIGS. 37-38 illustrate the assemblable pump system 100 including the first pump 102 provided with the coupler 114 that is a pump-receiving coupler 3700 fixedly attached to the driveline 110, and the second pump 122 provided with an abutment stop, such as a node 3702, provided on the driveline 130 thereof, according to one or more embodiments. The pump-receiving coupler 3700 defines an opening 3704 that is sized and shaped to slidably receive (as shown by the double-headed arrow) the driveline 130 of the second pump 122 therein. The pump-receiving coupler 3700 is configured to abut the node 3702 (which in this embodiment replaces the bulge 3102) in order to stop the relative sliding of the first and second pumps 102, 122 upon actuation. Optionally, as illustrated, the first and second pumping units 104, 124 may be provided with first and second magnetic connectors 4200, 4202, respectively, as described hereinafter. In this case, the first and second magnetic connectors 4200, 4202 are configured to magnetically connect together when the pump-receiving coupler 3700 and the node 3702 abuts or substantially abuts together.

The pump-receiving coupler 3700 being generally similar to the pump-receiving coupler 3102, the pump-receiving coupler 3700 will be briefly described hereinafter. It will be appreciated that a similar description applies to the pump-receiving couplers 3700 and 3102, with the necessary change(s), appreciable to the skilled addressee, having been made, if applicable.

As illustrated in FIG. 37, the assemblable pump system 100 is in the unassembled state with the first and second pumps 102, 122 contained in a sheath (which is shown in dashed lines as an environmental object) for delivery to an implantation site.

In this embodiment, the pump-receiving coupler 3700 is not configured to convert between an undeployed state and a deployed state. However, when the pump-receiving coupler 3700 is so sized and/or shaped that it cannot be contained in a sheath while retaining the capacity to couple with the second pump 122, the pump-receiving coupler 3700 is configured to convert between an undeployed state and a deployed state. To this end, as described for the pump-receiving coupler 3102, the pump-receiving coupler 3700 may be made of or coupled to a memory material, such as nitinol or silicone, that is overcomeably biased toward the deployed state. Alternatively, the pump-receiving coupler 3700 may be overcomeably spring-loaded toward the deployed state. When overcomeably biased or spring-loaded, the pump-receiving coupler 3700 is configured to be re-sheated for retrieval, as described for the pump-receiving coupler 3102. Still alternatively, the driveline 110 of the first pump 102 may include an actuation mechanism coupled to the pump-receiving coupler 3702 that is operable by an operator in such a way to convert the pump-receiving coupler 3702 between the undeployed and deployed states, when the assemblable pump system 100 is outside an optional sheath.

As illustrated in FIG. 38, the first and second pumps 102, 122 have been exited from the sheath (not shown), are delivered at the implantation site, and are now in the assembled state, as a result of the slidable actuation of at least one of the drivelines 110, 130 and relative movement of at least one of the first and second pumps 102, 122, as described herein. The node 3702 of the second pump 122 abuts the pump-receiving coupler 3700, preventing relative movement of the first and second pumping units 104, 124 by pushing the driveline 110 of the first pump 102 and/or pulling the driveline 130 of the second pump 122.

In embodiments, similar to the pump-receiving coupler 3100, the assemblable pump system 100 may be provided with the pump-receiving coupler 3700 in addition to be also provided with one or more magnet(s) (not shown), and/or the first and second electrical connectors 4400, 4402 (as described in relation to FIGS. 44-45; not shown in FIGS. 37-38). It will be appreciated that a similar description applies to the pump-receiving coupler 3700 and the pump-receiving coupler 3100, with the necessary change(s), appreciable to the skilled addressee, having been made, if applicable.

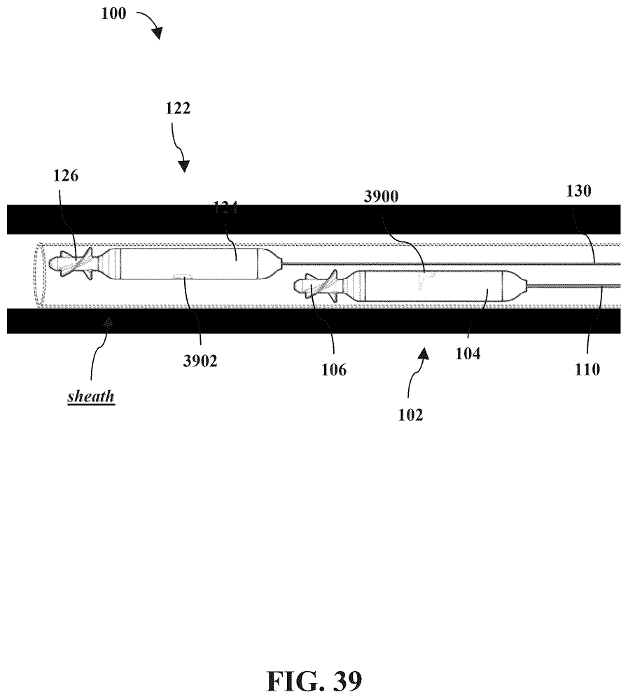

FIGS. 39-41 illustrate the assemblable pump system 100 including the first pump 102 provided with the first coupler 116 that is first physical connector, such as a male connector 3900, and the second pump 122 provided with the second coupler 226 that is second physical connector, such as a female coupler 3902, according to one or more embodiments. The male and female connectors 3900, 3902 are configured to cooperatively mate or cooperatively connect together by receiving the male connector into the female connector for assembling the first and second pumps 102, 122 together.

As illustrated in FIG. 39, the assemblable pump system 100 is in an unassembled state with the first and second pumps 102, 122 contained in a sheath (which is shown in dashed lines as an environmental object), and the male connector 3900 (shown in dashed lines as a way to see through the first pumping unit 104 through the first pumping unit 104) in an undeployed state for delivery to an implantation site. In particular, in the undeployed state, the male connector 3900 is pivoted toward the first pumping unit 104 so that the male connector 3900 so that to be contained or substantially contained inside the first pumping unit 104. As such, in the undeployed state, the cross-sectional size of the first pumping unit 104 is the same or substantially the same as if there would be no male connector 3900 provided thereto. Alternatively, the male connector 3900 may be configured to be translated inside and outside the first pumping unit 104 and have the same cross-sectional size in the undeployed state as when configured to pivot.

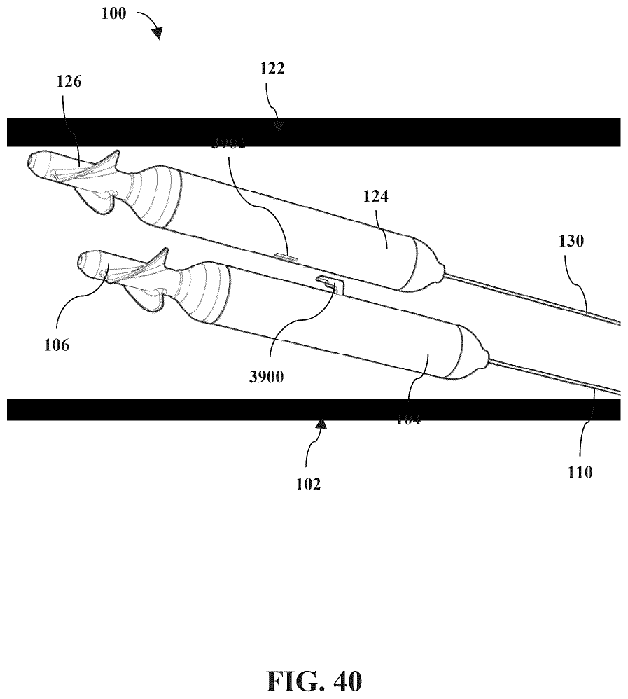

As illustrated in FIG. 40, the assemblable pump system 100 is still in the unassembled state. However, the first and second pumps 102, 122 have been exited from the sheath (not shown) and delivered at the implantation site. The first and second pumps 102, 122 are ready to be assembled together by manipulation of at least one of the drivelines 110, 130, respectively, as described herein. Further, the male connector 3900 is now in the deployed state. The male connector 3900 may convert between the undeployed state and the deployed state by being overcomeably spring-loaded toward the deployed state, such as by pivoting or by translating relative to the first pumping unit 104. Alternatively, the male connector 3900 may be made of or be coupled to a memory material, such as nitinol and/or silicone, that is overcomeably biased toward the deployed state.

In embodiments where the assemblable pump system 100 is transcatheterly deliverable, the male connector 3900, when overcomeably biased toward the deployed state, is configured to auto-convert from the undeployed state to the deployed state as the sheath releases the constraint imposed on the male connector 3900 while being exited therefrom for delivery. Conversely, the male connector 3900 may also be configured to convert back from the deployed state to the undeployed state as the assemblable pump system 100 is re-sheated for retrieval.

Alternatively, the driveline 110 of the first pump 102 may include an actuation mechanism coupled to the male connector 3900 that is operable by an operator in such a way to convert the male connector 3900 between the undeployed and deployed states, when the assemblable pump system 100 is outside an optional sheath.

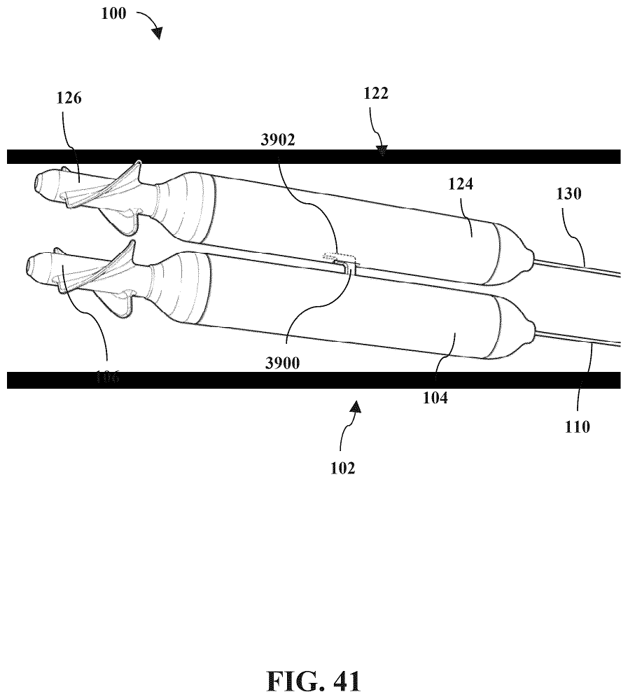

As illustrated in FIG. 41, the assemblable pump system 100 is delivered at the implantation site and is now in the assembled state, as a result of the slidable actuation of at least one of the drivelines 110, 130 and relative movement of at least one of the first and second pumps 102, 122, as described herein. The male connector 3900 of the first pump 102 engages and is received in the female connector 3902 of the second pump 122.

Whereas in this embodiment the first and second couplers 116, 226 are disclosed as being male and female connectors, it will be appreciated that the first and second couplers 116, 226 may in fact be any other type of physical connectors configured to cooperatively connect and/or mechanically connect together, either it be a removable connection or not. In particular, the first and second couplers 116, 226 include any physical connectors, physical fasteners, and cooperatively mating structures known in the art that are capable of intraluminally connecting together by actuation of one or more driveline(s) that is attached to at least one of the first and second couplers 116, 226.

For example, contemplated first and second couplers 116, 226 include cam latch connectors, compression latch connectors, slam latch connectors, toggle latch connectors, sliding latch connectors, magnetic latch connectors, hasp latch connectors, hook-loop connectors or fasteners, and the like. Also any of the first and second couplers 116, 226, including the contemplated latch connectors, may be coupled to a latch mechanism that an operator may operate to connect the first and second couplers 116, 226 together and/or to disconnect the first and second couplers 116, 226 form each other.

Still for example, the first and second couplers 116, 226 may also be configured to have complementary size and shape allowing them to physically connect together. The first coupler 116 may be the outer surface of the first pumping unit 104, and the second coupler 226 may be the outer surface of the second pumping unit 124. In this case, each of the outer surfaces may be provided with corresponding threads that mechanically inter-engage upon rotational actuation of the drivelines 110, 130 of the first and second pumps 102, 122, respectively.

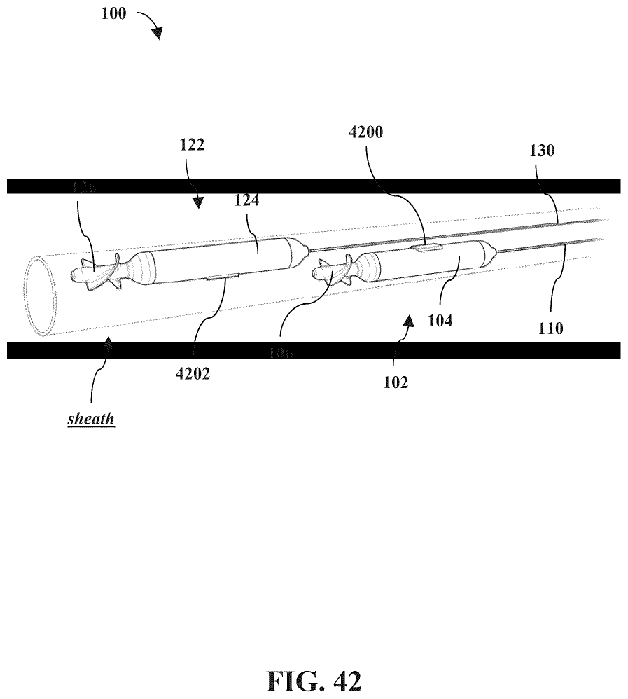

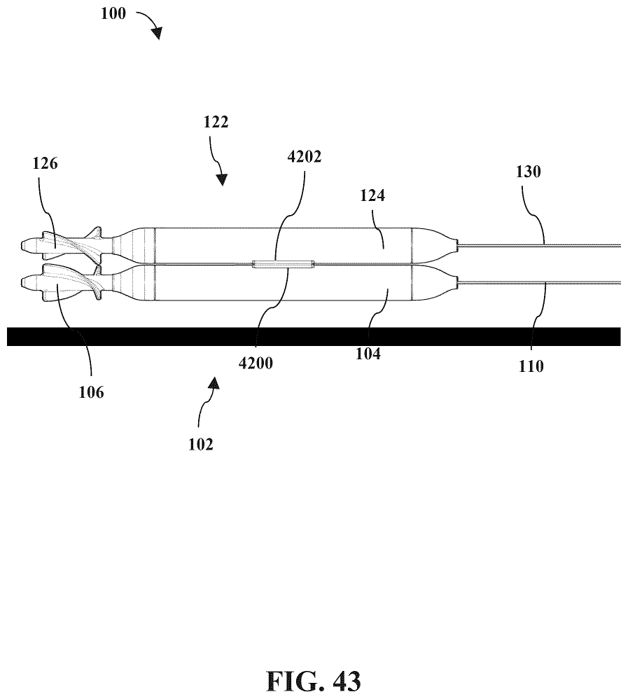

FIGS. 42-43 illustrate the assemblable pump system 100 including the first pump 102 provided with the first coupler 116 that is a first magnetic connector 4200, and the second pump 122 provided with the second coupler 226 that is a second magnetic connector 4202, according to one or more embodiments. The first and second magnetic connectors 4200, 4202 are configured to magnetically connect together for assembling the first and second pumps 102, 122 together.

As illustrated in FIG. 42, the assemblable pump system 100 is in an unassembled state with the first and second pumps 102, 122 contained in a sheath (which is shown in dashed lines as an environmental object) for delivery to an implantation site.

In this embodiment, as illustrated, the first and second magnetic connectors 4200, 4202 are not configured to convert between an undeployed state and a deployed state. However, when one or both of the first and second magnetic connectors 4200, 4202 is so sized and/or shaped that it cannot be contained in a sheath while retaining the capacity to magnetically connect the other one of the first and second magnetic connectors 4200, 4202, one or both of the first and the second magnetic connectors 4200, 4202 is/are configured to convert between an undeployed state and a deployed state. To this end, as described for the male and female connectors 3900, 3902, at least one of the first and second magnetic connectors 4200, 4202 may be convertible between the undeployed and deployed states by being overcomeably spring-loaded toward the deployed state, such as by pivoting or by translating relative to the first and second pumping units 104, 124, respectively. Alternatively, at least one of the first and second magnetic connectors 4200, 4202 may be made of or coupled to a memory material, such as nitinol and/or silicone, that is overcomeably biased toward the deployed state.

In embodiments where the assemblable pump system 100 is transcatheterly deliverable, one or both of the first and second magnetic connectors 4200, 4202, when overcomeably biased toward the deployed state, is/are configured to auto-convert from the undeployed state to the deployed state as the sheath releases the constraint imposed on the first and second magnetic connectors 4200, 4202 while being exited therefrom for delivery. Conversely, one or both of the first and second magnetic connectors 4200, 4202 may also be configured to convert back from the deployed state to the undeployed state as the assemblable pump system 100 is re-sheated for retrieval.

Alternatively, one or both of the drivelines 110, 130 of the first and second pumps 102, 122 may include an actuation mechanism coupled to the first and second magnetic connectors 4200, 4202, respectively, that is operable by an operator in such a way to convert one or both of the first and second magnetic connectors 4200, 4202 between the undeployed and deployed states, when the assemblable pump system 100 is outside an optional sheath.

As illustrated in FIG. 43, the first and second pumps 102, 122 have been exited from the sheath (not shown), are delivered at the implantation site, and are now in the assembled state, as a result of the slidable actuation of at least one of the drivelines 110, 130 and relative movement of at least one of the first and second pumps 102, 122, as described herein. The first and second magnetic connectors 4200, 4202 are magnetically connected together.

In embodiments, only one of the first and second magnetic connectors 4200, 4202 is provided to a corresponding one of the first and second pumps 102, 122, such as to one of the first and second pumping units 104, 124. In this case, the other one of the first and second pumps 102, 122 includes a ferromagnetic material that is configured to magnetically couple one of the first and second magnetic connectors 4200, 4202.

Whereas in this embodiment the assemblage pump system 100 is disclosed as having dedicated first and second magnetic connectors 4200, 4202, which may be temporary magnets, permanent magnets, and electromagnets, for assembling the first and second pumps 102, 122 together, it will be appreciated that the first and second magnetic connectors 4200, 4202 may in fact be the corresponding motor magnets of the first and second drive units 108, 128, respectively.

FIGS. 44-45 illustrate the assemblable pump system 100 including the first pump 102 provided with the first coupler 116 that is a first electrical connector 4400, and the second pump 122 provided with the second coupler 226 that is a second electrical connector 4402, according to one or more embodiments. The first and second electrical connectors 4400, 4402 are configured to electrically connect together, either it be removably or not, so that electricity may be transmitted between the first and second pumps 102, 122 when assembled. In particular, electricity that is delivered from a power source (not shown) to the second pump 122 by the driveline 130 may be transmitted to the first pump 102 by the connection of the first and second electrical connectors 4400, 4402 together. In this case, the optional driveline 110 of the first pump 102 may not include the electrical conductor 112.

Each of the first and second electrical connectors 4400, 4402 include a plurality of electrical pins, such as three electrical pins 4404, 4406, 4408 (shown for the first pump 102 in FIG. 44, and for the second pump 122 in FIG. 45) as illustrated. A fluid seal (not shown) may be provided to at least one of the first and second electrical connectors 4400, 4402 to provide a fluid-tight contact between the same and prevent blood from contacting the electrical connection pins 4404, 4406, 4408. A magnet (not shown) may also be provided to at least one of the first and second electrical connectors 4400, 4402, while the other one of the first and second electrical connectors 4400, 4402 may be provided with another magnet (not shown) or a ferromagnetic material (not shown) for magnetically coupling the magnet. The magnets may help in maintaining an electrical connection and/or a fluid-tight contact between the first and second electrical connectors 4400, 4402.

As illustrated in FIG. 44, the assemblable pump system 100 is in an unassembled state with the first and second pumps 102, 122 contained in a sheath (which is shown in dashed lines as an environmental object) for delivery to an implantation site.

In this embodiment, as illustrated, the first and second electrical connectors 4400, 4402 are not configured to convert between an undeployed state and a deployed state. However, when one of both of the first and second electrical connectors 4400, 4402 is so sized and/or shaped that it cannot be contained in a sheath while retaining the capacity to electrically connect the other one of the first and second electrical connectors 4400, 4402, one or both of the first and the second electrical connectors 4400, 4402 is/are configured to convert between an undeployed state and a deployed state.

The first and second electrical connectors 4400, 4402 being generally similar to the first and second magnetic connectors 4200, 4202 with respect to the overcomeable conversion between the undeployed and deployed states, including the actuation mechanism thereof, the first and second electrical connectors 4400, 4402 will not be further described herein in this regard for the sake of brevity. It will be appreciated that a similar description applies to the first and second electrical connectors 4400, 4402 and first and second magnetic connectors 4200, 4202 for this purpose, with the necessary change(s), appreciable to the skilled addressee, having been made, if applicable.

As illustrated in FIG. 45, the first and second pumps 102, 122 have been exited from the sheath (not shown), are delivered at the implantation site, and are now in the assembled state, as a result of the slidable actuation of at least one of the drivelines 110, 130 and relative movement of at least one of the first and second pumps 102, 122, as described herein. The first and second electrical connectors 4400, 4402 are electrically connected together.

In absence of any other connectors, the first and second electrical connectors 4400, 4402 may be configured not only to electrically connect the first and second pumps 102, 122 together, but also to assemble the first and second pumps 102, 122 together.

The first and second electrical connectors 4400, 4402 include any type of electrical connectors configured to cooperatively electrically connect together, either it be a removable electrical connection or not, that are capable of intraluminally electrically connecting together by actuation of one or more driveline(s) that is attached to at least one of them. For example, contemplated first and second couplers 116, 226 include male and female electrical connectors In embodiments, one or both of the first and second couplers 116, 226 is the first and second physical connectors, such as the male and female connectors 3900, 3902, the first and second magnetic connectors 4200, 4202, the first and second electrical connectors 4400, 4402, and any combination(s) thereof, respectively.

For example, the male connector 3900 may be combined with the first magnetic connector 4200 into a single connector component, and/or the female connector 3902 may be combined with the second magnetic connector 4202 into a single connector component. The male connector 3900 may be combined with the first electrical connector 4400 into a single connector component, and/or the female connector 3902 may be combined with the second electrical connector 4402 into a single connector component. The first magnetic connector 4200 may be combined with the first electrical connector 4400, and/or the second magnetic connector 4202 may be combined with the second electrical connector 4402. The male connector 3900 may be combined with the first magnetic connector 4200 and the first electrical connector 4400 into a single connector component, and/or the female connector 3902 may be combined with the second magnetic connector 4202 and the second electrical connector 4402 into a single connector component. When only one of the first and second pumps 102, 122 is provided with a corresponding one of the first and second magnetic connectors 4200, 4202, the other one of the first and second pumps 102, 122 is provided with a ferromagnetic material for magnetically coupling the corresponding one of the first and second magnetic connectors 4200, 4202.

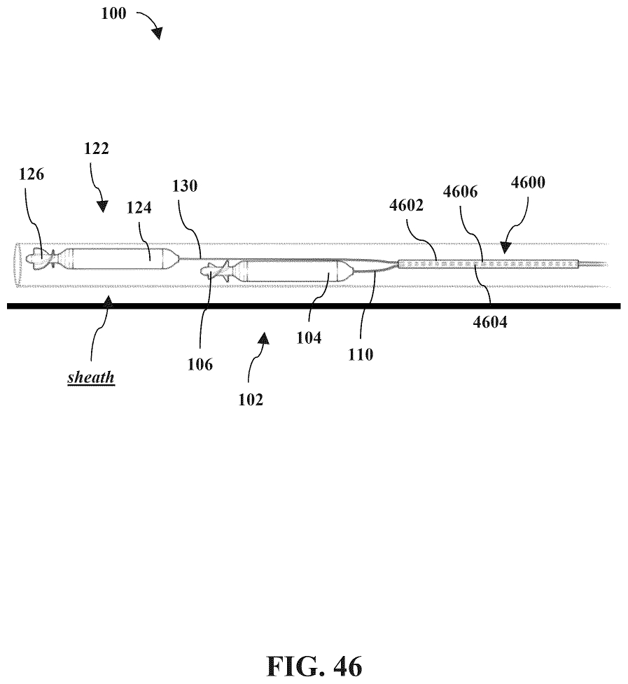

FIGS. 46-48 illustrate the assemblable pump system 100 including the first pump 102 provided with the driveline 110 thereof, the second pump 122 provided with the driveline 130 thereof, and a sleeve coupler 4600 configured to slidably receive at least one of the drivelines 110, 130, according to one or more embodiments. The sleeve coupler 4600 has an elongated body 4602 defining two longitudinal passageways 4604, 4606 (shown in dashed lines as a way to see through the sleeve coupler 4600), each sized and shaped to slidably receive a corresponding one of the drivelines 110, 130 of the first and second pumps 102, 122 therein. Alternatively, the sleeve coupler 4600 may define a single longitudinal passageway (not shown) that is sized and shaped to slidably receive both drivelines 110, 130 of the first and second pumps 102, 122 therein.

As illustrated in FIG. 46, the assemblable pump system 100 is in an unassembled state with the first and second pumps 102, 122 contained in a sheath (which is shown in dashed lines as an environmental object) for delivery to an implantation site.

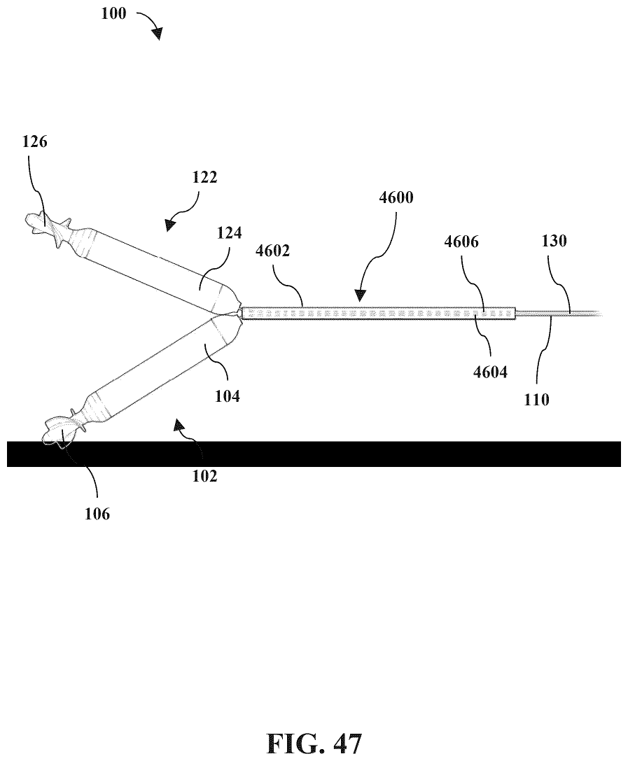

As illustrated in FIG. 47, the first and second pumps 102, 122 have been exited from the sheath (not shown), are delivered at the implantation site, and are now in the assembled state, as a result of the slidable actuation of the sleeve receiver 4600 and/or the drivelines 110, 130. In particular, slidably pushing the sleeve coupler 4600, such as by manipulating an extracorporeally-disposed portion thereof, toward the first and second pumping units 104, 124 brings the first and second pumping units into closer proximity from each other, ultimately resulting in the assembling of the first and second pumps 102, 122 upon abutment or substantial abutment of the sleeve coupler 4600 to the first and second pumping units 104, 124, as illustrated in FIG. 47. Alternatively, or additionally, to the slidable movement of the sleeve coupler 4600, the drivelines 110, 130 may by slidably pulled relative to the sleeve coupler 4600 to assemble the first and second pumps 102, 122 together.

In an embodiment, the sleeve coupler 4600 may be fixedly attached to the first pump 102, and may define a single longitudinal passageway (not shown) that is sized and shaped to receive the driveline 130 of the second pump 122 therein. Slidably pushing the attached sleeve coupler 4600, such as by manipulating an extracorporeally-disposed portion thereof, toward the second pumping unit 124 and/or pulling the driveline 130 of the second pump 122 that is received in the single longitudinal passageway brings the first and second pumping units 104, 124 into closer proximity from each other, ultimately resulting in the assembling of the first and second pumps 102, 122, as described hereinbefore.

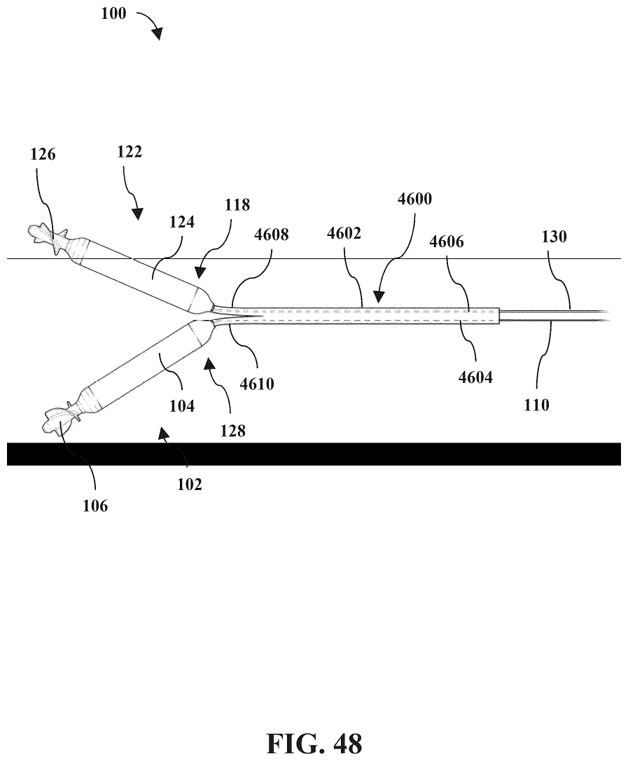

FIG. 48 illustrates the assemblable pump system 100 in the assembled state with the sleeve coupler 4600 provided with two arms 4800, 4802, each defining a portion of the longitudinal passageways 4604, 4606 that slidably receives corresponding drivelines 110, 130, according to one or more embodiments.

When assembled, the first and second pumping units 104, 124 are substantially parallelly aligned together such that the proximal end portions 118, 228 thereof converge together, as described hereinbefore and illustrated in FIGS. 47-48. The first and second pumping units 104, 124 may be provided with first and second couplers 116, 226 (not shown), such as the first and second magnetic connectors 4200, 4202 and/or the male and female connectors 3900, 3902, that are provided to corresponding proximal end portions 118, 228 of the first and second pumping units 104, 124, respectively. So proximally positioned, the first and second couplers 116, 226 may serve to assist in or help maintaining the first and second pumping units 104, 124 substantially parallelly aligned together such that the proximal end portions 118, 228 thereof converge together.

Alternatively, the first and second pumping units 104, 124 may be provided with corresponding first and second couplers 116, 226, such as the first and second magnetic connectors 4200, 4202 and/or the male and female connectors 3900, 3902, that are provided to corresponding distal end portions 120, 230 of the first and second pumping units 104, 124, respectively. So distally positioned, the first and second couplers 116, 226 may serve to assist in or help maintaining the first and second pumping units 104, 124 parallelly aligned together, as described hereinbefore.

The assemblable pump system 100 provided with the sleeve coupler 4600 is unassembled by slidably moving at least one of the drivelines 110, 130 relative to the sleeve coupler 4600 in such a way that the first and second pumping units 104, 124 are allowed to separate and transversally move away from each other.

Whereas the sleeve coupler 4600 are disclosed herein as having an elongated body 4602 that runs along a substantial portion of the drivelines 110, 130, it will be appreciated that the sleeve coupler 4600 may also be of a shorter length, such as to take the forms of one or more ring(s) that may be configured to slidably move to be put in place over at least one of the drivelines 110, 130 by a tool.

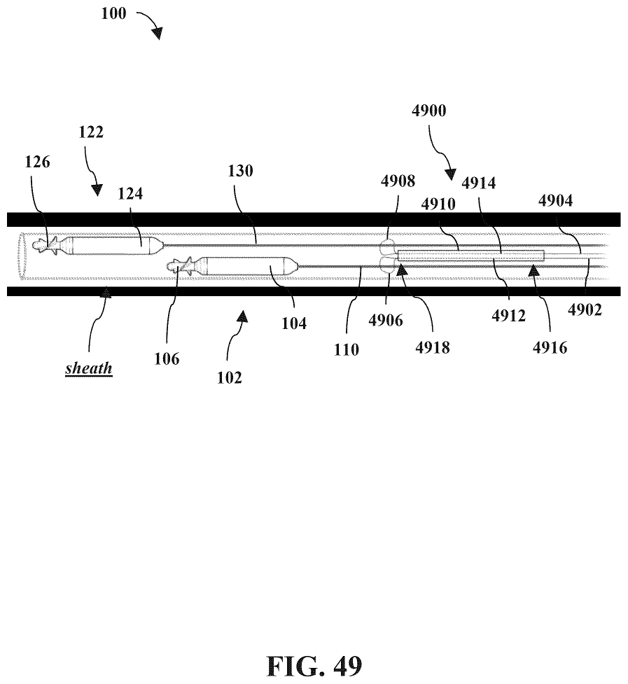

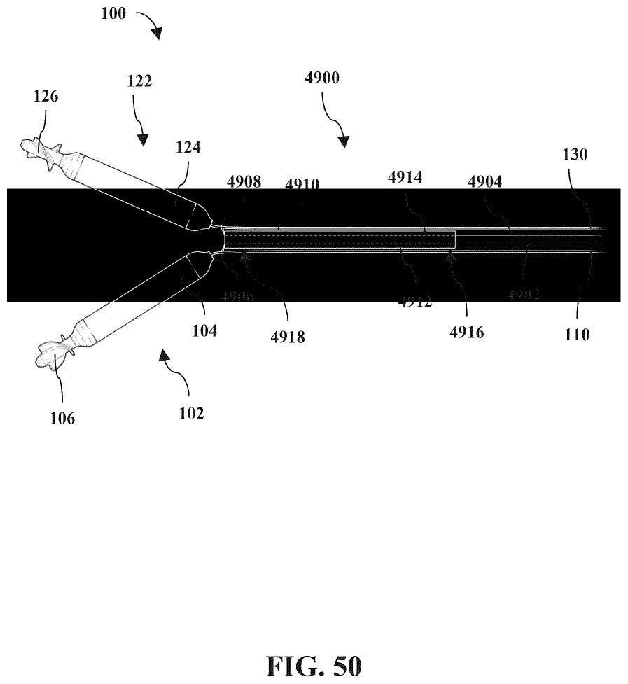

FIGS. 49-50 illustrate the assemblable pump system 100 including the first pump 102 provided with the driveline 110 thereof, the second pump 122 provided with the driveline 130 thereof, and a butterfly snare coupler 4900 provided with first and second snaring guide wires 4902, 4904 defining corresponding first and second snaring loops 4906, 4908, according to one or more embodiments. The butterfly snare coupler 4900 has an elongated body 4910 defining two longitudinal passageways 4912, 4914 (shown in dashed lines as a way to see through the butterfly snare coupler 4900), each sized and shaped to slidably receive a corresponding one of the first and second snaring guide wires 4902, 4904 therein. Alternatively, the butterfly snare coupler 4900 may define a single longitudinal passageway (not shown) that is sized and shaped to slidably receive both first and second snaring guide wires 4902, 4904 therein. Received in the corresponding longitudinal passageways 4912, 4914, the first and second snaring guide wires 4902, 4904 have corresponding manipulable end portions disposed at the proximal end portion 4916 of the butterfly snare coupler 4900, and have the first and second snaring loops 4906, 4908 thereof disposed at a distal end portion 4918 of the butterfly snare coupler 4900.

As illustrated in FIG. 49, the assemblable pump system 100 is in an unassembled state with the first and second pumps 102, 122 contained in a sheath (which is shown in dashed lines as an environmental object) for delivery to an implantation site. Each of the drivelines 110, 130 are received in the corresponding first and second snaring loops 4906, 4908, which are in the open state. In the open state, the drivelines 110, 130 may freely slidably move through the first and second snaring loops 4906, 4908, respectively, upon slidable actuation of the drivelines 110, 130 and/or the butterfly snare coupler 4900.

As illustrated in FIG. 50, the first and second pumps 102, 122 have been exited from the sheath (not shown), are delivered at the implantation site, and are now in the assembled state, as a result of the slidable actuation of the drivelines 110, 130 and/or the butterfly snare coupler 4900, and also of the transition of the first and second snaring loops 4906, 4908 from the open state to a close state. In particular, slidably pushing the butterfly snare coupler 4900, such as by manipulating an extracorporeally-disposed portion thereof, toward the first and second pumping units 104, 124 brings the first and second pumping units 102, 122 into closer proximity from each other. Alternatively, or additionally, to the slidable movement of the butterfly snare coupler 4900, the drivelines 110, 130 may by slidably pulled relative to the butterfly snare coupler 4900 to bring the first and second pumping units 102, 122 into closer proximity from each other. Upon abutment or substantial abutment of the butterfly snare coupler 4900 to the first and second pumping units 104, 124, the manipulable portions of the first and second snaring guide wires 4902, 4904 are pulled to transition the first and second snaring loops 4906, 4908 from the open state to the close state, resulting in the assembling of the first and second pumps 102, 122 together, as illustrated in FIG. 50. In the close state, the drivelines 110, 130 are no longer freely slidably movable through the first and second snaring loops 4906, 4908, respectively.

In an embodiment, the butterfly snare coupler 4900 may be fixedly attached to the first pump 102, and may define a single longitudinal passageway (not shown) that is sized and shaped to receive the second snaring guide wires 4904 therein, which second snaring loop 4908 slidably receive the driveline 130 of the second pump 122 therethrough. Slidably pushing the attached butterfly snare coupler 4900, such as by manipulating an extracorporeally-disposed portion thereof, toward the second pumping unit 124 and/or pulling the driveline 130 of the second pump 122 that is received through the second snaring guide wires 4904 brings the first and second pumping units 104, 124 into closer proximity from each other. Then, transitioning the second snaring loop 4908 from the open state to the close state slidably immobilize the driveline 130 of the second pump 122, ultimately resulting in the assembling of the first and second pumps 102, 122, as described hereinbefore.

The butterfly snare coupler 4900 being generally similar to the sleeve coupler 4600 with respect to relative alignment of the first and second pumping units 104, 124 in the assembled state, the butterfly snare coupler 4900 will not be further described herein in this regard for the sake of brevity. It will be appreciated that a similar description applies to the butterfly snare coupler 4900 and the sleeve coupler 4600 for this purpose, with the necessary change(s), appreciable to the skilled addressee, having been made, if applicable.

The assemblable pump system 100 provided with the butterfly snare coupler 4900 is unassembled by slidably moving at least one of the drivelines 110, 130 through the corresponding first and second snaring loops 4906, 4908, respectively, in such a way that the first and second pumping units 104, 124 are allowed to separate and transversally move away from each other.

As intended herein, the couplers 114, 134, 116, 226 may be any structures that are configured such that the first and second pumps 102, 122 can be converted from the assembled state to the unassembled state and/or from unassembled state to the assembled state by manipulation of corresponding driveline, without departing from the scope of the present disclosure. For example, the couplers 114, 134, 116, 126 may include a latch coupler, a cam latch coupler, a compression latch coupler, a slam latch coupler, a toggle latch coupler, a sliding latch coupler, a magnetic latch coupler, and/or a hasp latch coupler. Alternatively or additionally, the couplers 114, 134, 116, 226 may be any two geometrical shapes that are configured to cooperatively mate with each other.

Also, the first and second couplers 116, 226 may include first and second physical connectors, first and second magnetic connectors, first and second electrical connectors, and combination thereof, respectively. The first and second physical connectors are configured to physically engage to each other and/or to physically disengage from each other such that the first and second pumps 102, 122 may physically couple to each other and/or physically uncouple from each other. The first and second magnetic connectors are configured to magnetically engage to each other and/or to magnetically disengage from each other such that the first and second pumps 102, 122 may magnetically couple to each other and/or magnetically uncouple from each other. The first and second electrical connectors are configured to electrically connect to each other and/or to electrically disconnect from each other such that the first and second pumps 102, 122 may electrically couple to each other and/or electrically uncouple from each other.

For example, the first and second couplers 116, 226 may include first and second physical connectors as well as first and second electrical connectors. The first and second couplers 116, 226 may include first and second magnetic connectors as well as first and second electrical connectors. The first and second couplers 116, 226 may include first and second physical connectors, first and second magnetic connectors, as well as first and second electrical connectors. Alternatively, the first and second physical connectors, first and second magnetic connectors, first and second electrical connectors, and combination thereof may be provided elsewhere to the first and second pumps 102, 122, such as to the first and second pumping units 104, 124 and/or to the drivelines 110, 130, respectively.

As intended herein, the drivelines 110, 130 may include guide wires, wires, catheters, cables, snares, and equivalent structures configured or not to transmit power, such as electric power (e.g., electrical wires, electrical cables, and equivalent structure having an electrical conductor configured for this purpose) and/or mechanic power (e.g., driveshaft), without departing from the scope of the present disclosure.

As intended herein, the first and second pumps 102, 122 may be any pumps, including any blood or heart pumps, such as axial-flow pumps, positive-displacement pumps, and centrifugal pumps The first and second impellers 106, 126 may be any impellers, such an axial impellers, a peripheral impellers, a mixed flow impellers, or a radial impellers. Each of the first and second pumps 102, 122 may include a respective shroud configured to house the corresponding first and second impellers 106, 126 therein.

Although the assemblable pump system 100 may include components that require power to be operated (which may also be referred to herein as “powerable components” or “powerable medical devices”), namely the first and second pumps 102, 122 as disclosed herein, the assemblable pump system 100 may also include components that are not operable and thus that do not require power for operation (which may also be referred to herein as “non-powerable components” or “non-powerable medical devices”). In the case of non-powerable components, the drivelines 110, 130 are not configured to provide power to the first and second pumping units 104, 124 and thus may not include the electrical conductor 112, 132, respectively. The assemblable pump system 100 may be provided with powerable components, non-powerable components, and combination thereof. In any case, the powerable and non-powerable components are configured such that manipulation of at least one of the drivelines or corresponding structures thereof may be manipulated for converting the assemblable pump system 100 between the unassembled state to the assembled state and/or from the assembled state to the unassembled state.

Accordingly, the assemblable pump system 100 may include any components or medical devices, such as: blood flow influencing devices, balloon pump systems, occluders, endovascular prosthesis, implantable pacemakers, interatrial shunt devices, valve replacement systems, and/or valve plasty systems.

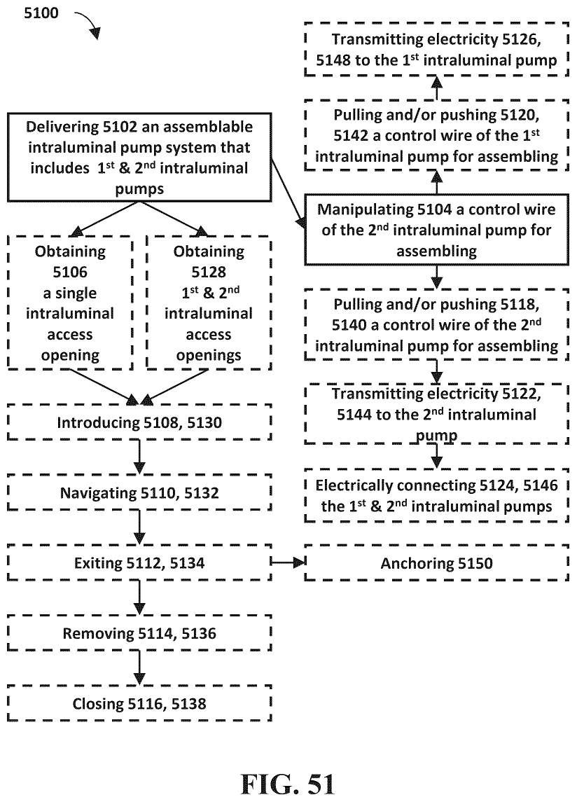

With reference now to FIGS. 51-54, methods of implanting and explanting the assemblable pump system 100 in vivo will be described, according to second and third aspects of this disclosure.

FIG. 51 schematically illustrates a method of implanting 5100 an assemblable pump system at an implantation site in a lumen of a subject, according to one or more embodiments. The method of implanting 5100 includes: delivering the assemblable pump system to the implantation site, at 5102. The assemblable pump system is provided with a first pump, which may or may not be provided with an optional driveline thereof, and a second pump, which has a driveline thereof configured to attach thereto and project therefrom. The first and second pumps are configured to intraluminally assemble together via a pump-to-pump coupling between the first and second pumps. The method of implanting 5100 also includes: manipulating the driveline of the second pump to move the second pump and to intraluminally assemble the first and second pumps together, at 5104.

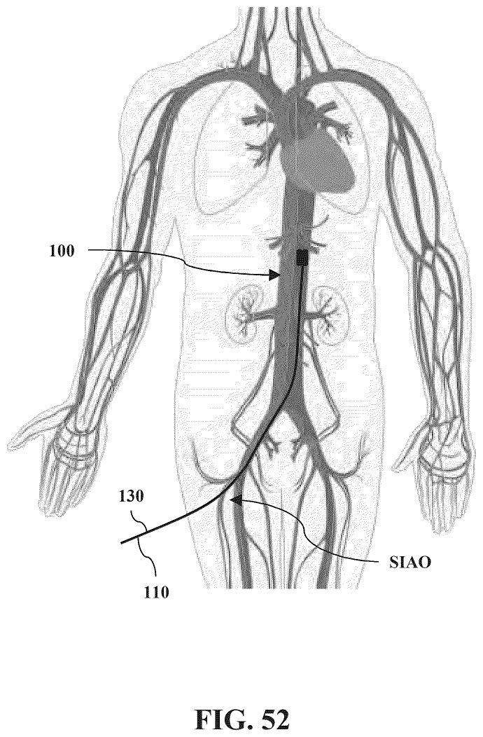

As schematically illustrated in FIG. 52, the assemblable pump system 100 may be delivered to the implantation site and operated via a single access opening SIAO (also referred to herein as “intracorporeal body access”), according to one or more embodiments. In this case, the first and second pumps are routed to the implantation site through the single access opening SIAO, which as illustrated receives the optional driveline of the first pump and the driveline of the second pump therethrough.

Delivering the assemblable pump system may include: obtaining the single access opening, such as by using the Seldinger technique, for delivering the first and second pumps therethrough to the implantation site, at 5106.

Delivering the assemblable pump system may further include: introducing an optional sheath, containing at least partially the first and second pumps therein, in the lumen through the single access opening SIAO, at 5108; navigating the optional sheath in the lumen up to the implantation site thereof, at 5110; exiting (or promoting the exit of) the first and second pumps from the optional sheath at the implantation site at 5112; and removing the optional sheath from the lumen and through the single access opening SIAO, at 5114.

Delivering the assemblable pump system may further include: closing the single access opening on the driveline of the second pump, at 5116.

Manipulating the driveline of the second pump may include: pulling and/or pushing the driveline of the second pump, as described herein, through the single access opening SIAO so that the first and second pumping units closer from each other to intraluminally assemble the first and second pumps together, at 5118.

In embodiments where the first pump is provided with the driveline thereof, the method of implanting 5100 may further include: pulling and/or pushing the driveline of the first pump, as described herein, through the single access opening SIAO so that the first and second pumping units get closer from each other to intraluminally assemble the first and second pumps together, at 5120.

Manipulating the drivelines of the first and second pumps respectively, may include: manipulating a portion of the drivelines of the first and second pumps, respectively, that is disposed extracorporeally relative to the single access opening SIAO. Alternatively, a portion of the drivelines of the first and second pumps, respectively, that is disposed intracorporeally (but not intraluminally) relative to the single access opening SIAO may be manipulated. Still alternatively, a portion of the drivelines of the first and second pumps, respectively, that is disposed intraluminally relative to the single access opening SIAO may be manipulated.

The method of implanting 5100 may further include: transmitting electricity to the second pump via the driveline thereof that is disposed through the single access opening SIAO for operating the second pump, at 5122.

Manipulating the driveline of the second pump through the single access opening SIAO may further include: electrically connecting the first and second pumps together to transmit electricity between the first and second pumps for operating the first pump, when the first and second pumps are assembled together, at 5124.

In embodiments where the first pump is provided with the driveline thereof, the method of implanting 5100 may further include: transmitting electricity to the first pump via the driveline thereof disposed through the single access opening SIAO for operating the first pump.

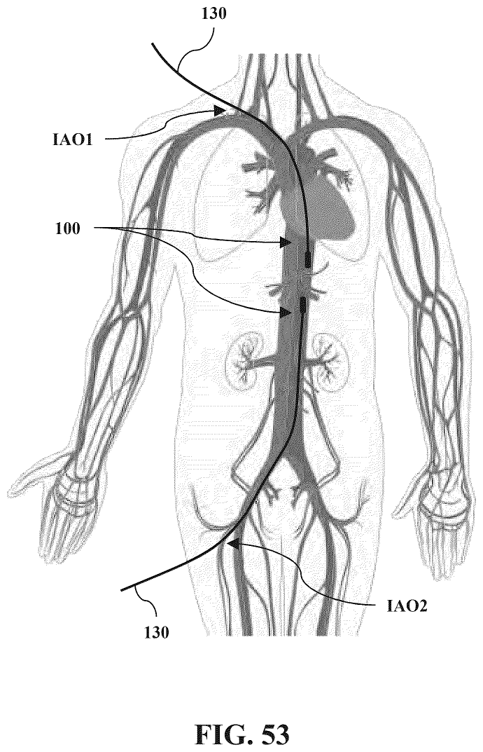

As schematically illustrated in FIG. 53, the assemblable pump system may be delivered to the implantation site and operated via a first access opening IAO1 (also referred to herein as “first intracorporeal body access”) and a second access opening IAO2 (also referred to herein as “second intracorporeal body access”), according to one or more embodiments. In this case, the first pump is routed to the implantation site through the first access opening IAO1, while the second pump is routed to the implantation site through the second access opening IAO2. As illustrated, the first access opening IAO1 receives the optional driveline of the first pump therethrough, and the second access opening IAO2 receives the driveline of the second pump therethrough. The first and second access openings IAO1, IAO2 may be located on the same lumen or on different lumens. For example, the first access openings IAO1 may provide intraluminal access to the femoral vessel or femoral artery, while the second access openings IAO2 may provide intraluminal access to the subclavian/axillary vessel or subclavian/axillary artery.

Delivering the assemblable pump system may include: obtaining the first and second access openings IAO1, AIO2 for delivering the corresponding first and second pumps therethrough to the implantation site, at 5128. For example, the first and second access openings IAO1, IAO2 may be obtained using the Seldinger technique.

Delivering the assemblable pump system may further include: introducing optional first and second optional sheathes, containing at least partially the first and second pumps therein, respectively, in the lumen through the corresponding first and second access openings IAO1, IAO2, at 5130; navigating the optional first and second sheathes in the lumen up to the implantation site thereof, at 5132; exiting the first and second pumps from the optional first and second sheathes, respectively, at the implantation site thereof, at 5134; removing the optional first and second sheathes from the lumen through the corresponding first and second access openings IAO1, IAO2, at 5136, and closing the first and second access openings IAO1, AIO2, at 5138.

Manipulating the driveline of the second pump may include: pulling and/or pushing the driveline of the second pump, as described herein, through the second access opening IAO2 so that the first and second pumping units get closer from each other to intraluminally assemble the first and second pumps together, at 5140.

In embodiments where the first pump is provided with the driveline thereof, the method of implanting 5100 may further include: pulling and/or pushing the driveline of the first pump, as described herein, through the first access opening IAO1 so that the first and second pumping units get closer from each other to intraluminally assemble the first and second pumps together, at 5142.

Manipulating the drivelines of the first and second pumps, respectively, may include: manipulating a portion of the drivelines of the first and second pumps, respectively, that is disposed extracorporeally relative to the corresponding first and second access openings AIO1, AIO2. Alternatively, a portion of the drivelines of the first and second pumps, respectively, that is disposed intracorporeally (but not intraluminally) relative to the corresponding first and second access openings AIO1, AIO2 may be manipulated. Still alternatively, a portion of the drivelines of the first and second pumps, respectively, that is disposed intraluminally relative to the corresponding first and second access openings AIO1, AIO2 may be manipulated.

The method of implanting 5100 may further include: transmitting electricity to the second pump via the driveline thereof disposed through the second access opening IAO2 for operating the second pump, at 5144.

Manipulating the driveline of the second pump includes: electrically connecting the first and second pumps together to transmit electricity between the first and second pumps for operation, when assembled together, at 5146.

The method of implanting 5100 may further include: transmitting electricity to the first pump via the driveline thereof disposed through the first access opening IAO1 for operating the first pump, at 5148.

Manipulating the drivelines of the first and second pumps, respectively, through the corresponding first and second access openings IAO1, AIO2 may further include: electrically connecting the first and second pumps together to transmit electricity between the first and second pumps for operation, when the first and second pumps are assembled together.

In embodiments where the first pump is provided with the driveline thereof, the method of implanting 5100 may further include: transmitting electricity to the first and second pumps via the drivelines thereof, respectively, that are disposed through the corresponding first and second access openings IAO1, AIO2 for operating the first and second pumps.

Alternatively, each of the first and second pump(s) may be operated by a respective drive shaft, which is provided to the drivelines, respectively, and disposed through the single access opening SIAO or the first and/or second access openings IAO1, AIO2. The drive shaft is coupled to a respective motor and configured to operate the first and/or second pump(s). A cooling fluid may also be provided to the drivelines for maintaining an appropriate operating temperature of the drivelines.

Still alternatively, the assemblable pump system may be delivered non-transcatheterly by pulling or pushing the drivelines of the first and second pumps through the single access opening or the first and/or second access openings IAO1, AIO2, along one or more lumen. In particular, the first and second pumps may be pushed by using guide wires of various stiffnesses or structural rigidities, as described hereinbefore. Alternatively, or additionally, the first and second pumps may be pushed or pulled while being rolled over a guide wire, as also described hereinbefore.

The method of implanting 5100 may further include: anchoring the assemblable pump system at the implantation site, at 5150.

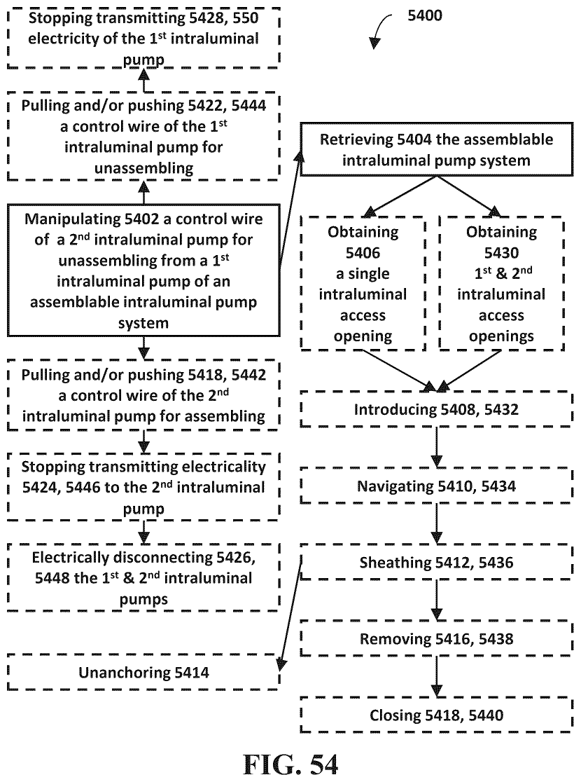

FIG. 54 schematically illustrates a method of explanting 5400 the assemblable pump system, which includes the first pump and the second pump, from the implantation site in a lumen of a subject, according to one or more embodiments. The method of explanting 5400 includes: manipulating the driveline of the second pump to move the second pump and to intraluminally unassemble the second pump from the first pump, which is coupled to the second pump via a pump-to-pump coupling, at 5402. The assemblable pump system is provided with the first pump, which may or may not be provided with the optional driveline thereof, and the second pump, which has the driveline thereof configured to attach thereto and project therefrom. The method of explanting 5400 also includes: retrieving the assemblable pump system, which is in the unassembled state, from the implantation site, at 5404.

Referring back to FIG. 52, the assemblable pump system may be implanted to the implantation site and operated via a single access opening SIAO. In this case, the first and second pumps are routed to the implantation site through the single access opening SIAO, which as illustrated receives the optional driveline of the first pump and the driveline of the second pump therethrough.

Retrieving the assemblable pump system may include: obtaining the single access opening SIAO, such as by using the Seldinger technique, for retrieving the first and second pumps therethrough from the implantation site, at 5406.

Retrieving the assemblable pump system may further include: introducing an optional sheath in the lumen through the single access opening SIAO, at 5408; navigating the optional sheath in the lumen up to the implantation site thereof, at 5410; sheathing at least partially the first and second pumps, at 5412; unanchoring the assemblable pump system from the implantation site, at 5414; removing the optional sheath, containing at least partially the first and second pumps therein, from the lumen and through the single access opening SIAO, at 5416, and closing the single access opening SIAO, at 5118.

Manipulating the driveline of the second pump may include: pulling and/or pushing the driveline of the second pump, as described herein, through the single access opening SIAO so that the first and second pumping units move away from each other to intraluminally unassemble the first and second pumps from each other, at 5420.

In embodiments where the first pump is provided with the driveline thereof, the method of explanting 5400 may further include: pulling and/or pushing the driveline of the first pump, as described herein, through the single access opening SIAO so that the first and second pumping units move away from each other to intraluminally unassemble the first and second pumps from each other, at 5422.

Manipulating the drivelines of the first and second pumps, respectively, may include: manipulating a portion of the drivelines of the first and second pumps, respectively, that is disposed extracorporeally relative to the single access opening SIAO. Alternatively, a portion of the drivelines of the first and second pumps, respectively, that is disposed intracorporeally (but not intraluminally) relative to the single access opening SIAO may be manipulated. Still alternatively, a portion of the drivelines of the first and second pumps, respectively, that is disposed intraluminally relative to the single access opening SIAO may be manipulated.

The method of explanting 5400 may further include: stopping transmitting electricity to the second pump via the driveline thereof disposed through the single access opening SIAO, at 5424.

Manipulating the driveline of the second pump through the single access opening SIAO may further include: electrically disconnecting the first and second pumps from each other to stop transmitting electricity between the first and second pumps, at 5426.

In embodiments where the first pump is provided with the driveline thereof, the method of explanting 5400 may further include: stopping transmitting electricity to the first pump via the driveline thereof disposed through the single access opening SIAO, at 5428.

Referring back to FIG. 53, the assemblable pump system may be implanted to the implantation site and operated via a first access opening IAO1 and a second access opening IAO2. In this case, the first pump is routed to the implantation site through the first access opening IAO1, while the second pump is routed to the implantation site through the second access opening IAO2. As illustrated, the first access opening IAO1 receives the optional driveline of the first pump therethrough, and the second access opening IAO2 receives the driveline of the second pump therethrough. The first and second access openings IAO, IAO2 may be located on the same lumen or on different lumens. For example, the first access openings IAO1 may provide intraluminal access to the femoral vessel or femoral artery, while the second access openings IAO2 may provide intraluminal access to the subclavian/axillary vessel or subclavian/axillary artery.

Retrieving the assemblable pump system may include: obtaining the first and second access openings IAO1, AIO2 for retrieving the corresponding first and second pumps therethrough from the implantation site, at 5430. For example, the first and second access openings IAO1, IAO2 may be obtained using the Seldinger technique.

Retrieving the assemblable pump system may further include: introducing optional first and second optional sheathes in the lumen through the corresponding first and second access openings IAO1, IAO2, at 5432; navigating the optional first and second sheathes in the lumen up to the implantation site thereof, at 5434; sheathing the first and second pumps in the optional first and second sheathes, respectively, at 5436; removing the optional first and second sheathes from the lumen through the corresponding first and second access openings IAO1, IAO2, at 5438; and closing the first and second access openings IAO1, IAO2, at 5440.

Manipulating the driveline of the second pump may include: pulling and/or pushing the driveline of the second pump, as described herein, through the second access opening IAO2 so that the first and second pumping units move away from each other to intraluminally unassemble the first and second pumps from each other, at 5442.

In embodiments where the first pump is provided with the driveline thereof, the method of explanting 5400 may further include: pulling and/or pushing the driveline of the first pump, as described herein, through the first access opening IAO1 so that the first and second pumping units move away from each other to intraluminally unassemble the first and second pumps from each other, at 5444.

Manipulating the drivelines of the first and second pumps, respectively, may include: manipulating a portion of the drivelines of the first and second pumps, respectively, that is disposed extracorporeally relative to the corresponding first and second access openings AIO1, AIO2, at MMM16. Alternatively, a portion of the drivelines of the first and second pumps, respectively, that is disposed intracorporeally (but not intraluminally) relative to the corresponding first and second access openings AIO1, AIO2 may be manipulated. Still alternatively, a portion of the drivelines of the first and second pumps, respectively, that is disposed intraluminally relative to the corresponding first and second access openings AIO1, AIO2 may be manipulated.

The method of explanting 5400, further includes: stopping transmitting electricity to the second pump via the driveline thereof disposed through the second access opening IAO2 for stopping operating the second pump at 5446.

Manipulating the driveline of the second pump includes: electrically disconnecting the first and second pumps from each other to stop transmitting electricity between the first and second pumps, when unassembled from each other, at 5448.

The method of explanting 5400, further includes: stopping transmitting electricity to the first pump via the driveline thereof disposed through the first access opening IAO1 for stopping operating the first pump, at 5550.

1. An assemblable pump system, comprising:
a first pump; and
a second pump including a driveline, the second pump being configured to intraluminally assemble to the first pump via a pump-to-pump coupling between the second pump and the first pump, the driveline being further configured to move the second pump to intraluminally assemble the second pump and the first pump together.

2. The system according to claim 1, wherein the first pump comprises a first pumping unit, and the second pump comprises a second pumping unit, the pump-to-pump coupling comprising a unit-to-unit coupling between the first pumping unit and the second pumping unit.

3. The system according to claim 1, wherein the first pump comprises a pumping unit, the pump-to-pump coupling comprising a unit-to-driveline coupling between the pumping unit of the first pump and the driveline of the second pump.

4. The system according to claim 1, wherein the first pump comprises a driveline.

5. The system according to claim 4, wherein the pump-to-pump coupling comprises a driveline-to-driveline coupling between the driveline of the first pump and the driveline of the second pump.

6. The system according to claim 4, wherein the driveline of the first pump is configured to removably attach to the first pump.

7. The system according to claim 4, wherein the driveline of the first pump is configured to screw to the first pump.

8. The system according to claim 4, wherein the driveline of the first pump is configured to snare the first pump.

9. The system according to claim 4, wherein the driveline of the first pump comprises an electrical conductor configured to transmit electricity from a power source to the first pump for operating the same.

10. The system according to claim 4, wherein the driveline of the first pump is configured to slidably move the first pump to intraluminally assemble the first pump and the second pump together.

11. The system according to claim 4, wherein the driveline of the first pump is configured to be extraluminally manipulated to move the first pump to intraluminally assemble the first pump and the second pump together.

12. The system according to claim 4, further comprising a sleeve coupler configured to attach to the driveline of the first pump and to slidably receive the driveline of the second pump therein.

13. The system according to claim 4, further comprising a sleeve coupler configured to slidably receive the driveline of the first pump and the second driveline of the second pump therein.

14. The system according to claim 1, further comprising a coupler associated with one of the first pump and the second pump, the coupler being configured to slidably receive the other one of the first pump and the second pump therein for intraluminally assembling the first pump and the second pump together.

15. The system according to claim 14, wherein the coupler comprises a pump-receiving coupler defining an opening sized and shaped to receive the other one of the first pump and the second pump therein.

16. The system according to claim 15, wherein the coupler is a latch coupler.

17. The system according to claim 1, further comprising a first physical connector associated with the first pump, and a second physical connector associated with the second pump, the first physical connector and the second physical connector being configured to couple together for intraluminally assembling the first pump and the second pump together.

18. The system according to claim 17, wherein the first physical connector comprises one of a male connector and a female connector, and the second physical connector comprises the other one of the male connector and the female connector.

19. The system according to claim 17, wherein the first physical connector is a first latch connector, and the second physical connector is a second latch connector.

20. The system according to claim 1, further comprising a first magnetic connector associated with the first pump, and a second magnetic connector associated with the second pump, the first magnetic connector and the second magnetic connector being configured to magnetically couple together for intraluminally assembling the first pump and the second pump together.

21. The system according to claim 20, wherein the first magnetic connector comprises a first drive unit magnet, and the second magnetic connector comprises a second drive unit magnet.

22. The system according to claim 1, further comprising a first electrical connector associated with the first pump, and a second electrical connector associated with the second pump, the first electrical connector and the second electrical connector being configured to electrically connect together for electrically connecting the first pump and the second pump together.

23. The system according to claim 22, wherein at least one of the first electrical connector and the second electrical connector comprises a fluid seal configured to provide a fluid tight contact upon electrical connection therebetween.

24. The system according to claim 15, wherein the coupler has an undeployed state for transcatheter delivery of at least one of the first pump and the second pump intraluminally, and a deployed state for intraluminally assembling the second pump and the first pump together.

25. The system according to claim 1, wherein the driveline of the second pump is configured to removably attach to the second pump.

26. The system according to claim 1, wherein the driveline of the second pump is configured to screw to the second pump.

27. The system according to claim 1, wherein the driveline of the second pump is configured to snare the second pump.

28. The system according to claim 1, wherein the driveline of the second pump comprises an electrical conductor configured to transmit electricity from a power source to the second pump for operating the same.

29. The system according to claim 1, wherein the driveline of the second pump is configured to slidably move the second pump to intraluminally assemble the second pump and the first pump together.

30. The system according to claim 1, wherein the driveline of the second pump is configured to be extraluminally manipulated to move the second pump to intraluminally assemble the second pump and the first pump together.

31. The system according to claim 1, further comprising an anchor attached to at least one of the first pump and the second pump, the anchor being configured to engage a lumen wall for intraluminally anchoring the first pump and the second pump.

32. The system according to claim 31, wherein the anchor is releasably attached to at least one of the first pump and the second pump.

33. The system according to claim 1, wherein each of the first pump and the second pump is an intravascular pump.
